SEQUENCE LISTING (36 sequences)

SEQ ID NO: 1 - protein, 14 aa, Artificial Sequence
  thrombin peptide derivative
RGDACXGDSGGPXV

SEQ ID NO: 2 - protein, 23 aa, Artificial Sequence
  thrombin peptide derivative
AGYKPDEGKRGDACXGDSGGPXV

SEQ ID NO: 3 - protein, 23 aa, Artificial Sequence
  thrombin peptide derivative
AGYKPDEGKRGDACEGDSGGPFV

SEQ ID NO: 4 - protein, 14 aa, Artificial Sequence
  thrombin peptide derivative
RGDAXXGDSGGPXV

SEQ ID NO: 5 - protein, 23 aa, Artificial Sequence
  thrombin peptide derivative
AGYKPDEGKRGDAXXGDSGGPXV

SEQ ID NO: 6 - protein, 23 aa, Artificial Sequence
  thrombin peptide derivative
AGYKPDEGKRGDACEGDSGGPFV

SEQ ID NO: 7 - protein, 10 aa, Artificial Sequence
  thrombin peptide derivative
EGKRGDACEG

SEQ ID NO: 8 - protein, 22 aa, Artificial Sequence
  thrombin peptide derivative
KGSPTVTFTGIPCFPFIRLVTS

SEQ ID NO: 9 - protein, 10 aa, Artificial Sequence
  thrombin peptide derivative
TFTGIPSFPF

SEQ ID NO: 10 - protein, 23 aa, Artificial Sequence
  thrombin peptide derivative
RPMFGLLPFAPLRTLPLSPPGKQ

SEQ ID NO: 11 - protein, 10 aa, Artificial Sequence
  thrombin peptide derivative
KPFAPLRTLP

SEQ ID NO: 12 - protein, 622 aa, Homo sapiens [sequence omitted]

SEQ ID NO: 13 - protein, 12 aa, Artificial Sequence
  thrombin peptide derivative
DXCXGDSGGPXV

SEQ ID NO: 14 - protein, 10 aa, Artificial Sequence
  serine esterase conserved sequence
CEGDSGGPFV

SEQ ID NO: 15 - protein, 10 aa, Artificial Sequence
  serine esterase conserved sequence
CXGDSGGPXV

SEQ ID NO: 16 - protein, 4 aa, Artificial Sequence
  thrombin peptide
RGDA

SEQ ID NO: 17 - protein, 14 aa, Artificial Sequence
  thrombin peptide derivative
RGDACEGDSGGPFV

SEQ ID NO: 18 - protein, 33 aa, Artificial Sequence
  thrombin peptide derivative
DNMFCAGYKPDEGKRGDACEGDSGGPFVMKSPF

SEQ ID NO: 19 - protein, 33 aa, Artificial Sequence
  thrombin peptide derivative
DNMFCAGYKPDEGKRGDACXGDSGGPXVMKSPF

SEQ ID NO: 20 - protein, 23 aa, Artificial Sequence
  thrombin peptide derivative
AGYKPDEGKRGDAXEGDSGGPFV

SEQ ID NO: 21 - protein, 23 aa, Artificial Sequence
  thrombin peptide derivative
AGYKPDEGKRGDAAEGDSGGPFV

SEQ ID NO: 22 - protein, 23 aa, Artificial Sequence
  thrombin peptide derivative
AGYKPDEGKRGDAAEGDSGGPFV

SEQ ID NO: 23 - protein, 33 aa, Artificial Sequence
  thrombin peptide derivative
DNMFXAGYKPDEGKRGDAXEGDSGGPFVMKSPF

SEQ ID NO: 24 - protein, 33 aa, Artificial Sequence
  thrombin peptide derivative
DNMFXAGYKPDEGKRGDAXXGDSGGPXVMKSPF

SEQ ID NO: 25 - protein, 33 aa, Artificial Sequence
  thrombin peptide derivative
DNMFAAGYKPDEGKRGDAAEGDSGGPFVMKSPF

SEQ ID NO: 26 - protein, 33 aa, Artificial Sequence; identical to SEQ ID NO: 25
  thrombin peptide derivative

SEQ ID NO: 27 - protein, 33 aa, Artificial Sequence
  thrombin peptide derivative
AGYKPDEGKRGDACEGDSGGPFVMKSPFNNRWY

SEQ ID NO: 28 - protein, 33 aa, Artificial Sequence
  thrombin peptide derivative
AGYKPDEGKRGDACXGDSGGPXVMKSPFNNRWY

SEQ ID NO: 29 - protein, 33 aa, Artificial Sequence; identical to SEQ ID NO: 27
  thrombin peptide derivative

SEQ ID NO: 30 - protein, 22 aa, Artificial Sequence
  complementary peptide of domain of thrombin
KGSPTVTFTGIPCFPFIRLVTS

SEQ ID NO: 31 - protein, 22 aa, Artificial Sequence
  complementary peptide of domain of thrombin
KGSPTVTFTGIPSFPFIRLVTS

SEQ ID NO: 32 - protein, 10 aa, Artificial Sequence
  complementary peptide of domain of thrombin
TFTGIPSFPF

SEQ ID NO: 33 - protein, 23 aa, Artificial Sequence
  complementary peptide of domain of thrombin
RPMFGLLPFAPLRTLPLSPPGKQ

SEQ ID NO: 34 - protein, 10 aa, Artificial Sequence
  complementary peptide of domain of thrombin
LPFAPLRTLP

SEQ ID NO: 35 - protein, 14 aa, Artificial Sequence
  thrombin peptide derivative
RGDAXEGDSGGPFV

SEQ ID NO: 36 - protein, 23 aa, Artificial Sequence
  thrombin peptide derivative
AGYKPDEGKRGDACXGDSGGPXV

(19) United States
(12) Patent Application Publication
(10) Pub. No.: US 2010/0303793 A1
(43) Pub. Date: Dec. 2, 2010

(54) COMBINATION THERAPY FOR CARDIAC REVASCULARIZATION AND CARDIAC REPAIR

(75) Inventors: Barbara Olszewska-Pazdrak, League City, TX (US); Darrell H. Carney, Dickinson, TX (US)

Correspondence Address: FOLEY & LARDNER LLP, 111 HUNTINGTON AVENUE, 26TH FLOOR, BOSTON, MA, 02199-7610, US

(73) Assignee: The Board of Regents, The University of Texas Syst

(21) Appl. No.: 12/595,343

(22) PCT Filed: Apr. 10, 2008
(86) PCT No.: PCT/US08/04612

§ 371 Date: Mar. 29, 2010

Related U.S. Application Data
(60) Provisional application No. 60/922,630, filed on Apr. 10, 2007.

(51) Int. Cl. A61K 38/46; A61K 38/18; A61P 35/00; A61P 9/00

(52) U.S. Cl. 424/94.6; 514/8.1; 514/8.2; 514/9.1

(57) ABSTRACT

An agonist of the non-proteolytically activated thrombin receptor and an angiogenic growth factor can be used in combination in methods of therapy to stimulate cardiac revascularization, to stimulate vascular endothelial cell proliferation, to stimulate vascular endothelial cell migration and to promote repair of cardiac tissue.

### RELATED APPLICATION

This application claims the benefit of U.S. Provisional Application No. 60/922,630, filed on Apr. 10, 2007. The entire teachings of the above application are incorporated herein by reference.

### BACKGROUND OF THE INVENTION

Cardiovascular diseases are generally characterized by an impaired supply of blood to the heart or other target organs. Myocardial infarction (MI) results from narrowed or blocked coronary arteries in the heart which starves the heart of needed nutrients and oxygen. When the supply of blood to the heart is compromised, cells respond by generating compounds that induce the growth of new blood vessels so as to increase the supply of blood to the heart. These new blood vessels are called collateral blood vessels. The process by which new blood vessels are induced to grow out of the existing vasculature is termed angiogenesis, and the substances that are produced by cells to induce angiogenesis are the angiogenic factors.

When heart muscle is deprived of oxygen and nutrients due to vascular occlusion, the heart muscle tissue becomes ischemic and loses its ability to contract. This loss of function may be restored by natural signals from the ischemic heart muscle that induce angiogenic revascularization through development of collateral vessels that bypass the occlusion. This revascularization or angiogenesis involves the stimulation of endothelial cell proliferation and migration and budding off of new blood vessels. In many cases, however, the natural signals are not sufficient to cause collateral vessel growth and the ischemic tissue can become fibrotic or necrotic. If this process is not reversed by procedures to open the occluded vessels or to induce growth of collateral blood vessels, the heart may become totally dysfunctional and require transplantation.

There is a need for pharmaceutical agents and treatments that can cause growth of new blood vessels in the heart and at other sites where improved circulation or repair of vascular tissue is needed.

### SUMMARY OF THE INVENTION

It is demonstrated herein, using a system to measure the response of human coronary artery endothelial cells to angiogenic factors, that TP508 treatment more than doubles the angiogenic potential of VEGF for endothelial cells, both under normoxic and hypoxic conditions. TP508 [the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3)] restores the ability of VEGF to activate eNOS (endothelial nitric oxide synthase), thereby increasing NO required to induce angiogenesis.

The invention encompasses methods of combination therapy, wherein an angiogenic factor and an NPAR agonist are both administered to a subject, in an amount and for a duration effective to promote cardiac tissue repair, cardiac revascularization, vascular endothelial cell proliferation and/or endothelial cell migration. The combination can be any combination of angiogenic factor and NPAR agonist.

Previously, the NPAR agonist TP508 was shown to stimulate proliferation and migration of endothelial cells and to stimulate angiogenesis when administered alone. Further, treatment with TP508 alone was effective in restoring the function of ischemic heart muscle and in stimulating myocardial revascularization. See U.S. Pat. No. 6,864,407, the contents of which are herein incorporated by reference in their entirety.

The invention encompasses methods of promoting cardiac tissue repair, methods of promoting cardiac revascularization, methods of promoting vascular endothelial cell proliferation, and methods of promoting vascular endothelial cell migration. The method, in each case, includes administering to the subject to be treated, a combination in a therapeutically effective amount, the combination comprising one or more angiogenic growth factors, and one or more agonists of the non-proteolytically activated thrombin receptor (NPAR agonists).

The invention also encompasses methods of promoting cardiac tissue repair, methods of promoting cardiac revascularization, methods of promoting vascular endothelial cell proliferation, and methods of promoting vascular endothelial cell migration, wherein the method comprises administering to the subject in need of one or more of these effects a combination in a therapeutically effective amount, the combination consisting essentially of an angiogenic growth factor and an agonist of the non-proteolytically activated thrombin receptor. In this method an angiogenic growth factor and an agonist of the non-proteolytically activated thrombin receptor (NPAR agonist) are administered in combination as the only therapeutically active agents.

The angiogenic growth factors in the methods can be any of the angiogenic growth factors known to those of skill in the art, for example, those listed in Tables 1 and 2. Preferred angiogenic growth factors are human. In some embodiments, the angiogenic growth factors are those of the VEGF family. In other embodiments, the angiogenic growth factor is human VEGF-A.

In some embodiments of the described methods, the NPAR agonist is a thrombin peptide derivative disclosed herein. More specifically, one thrombin peptide derivative comprises the amino acid sequence of Arg-Gly-Asp-Ala-Cys-X₁-Gly-Asp-Ser-Gly-Gly-Pro-X₂-Val (SEQ ID NO:1), or a C-terminal truncated fragment thereof comprising at least six amino acids. In specific embodiments, the thrombin peptide derivative comprises the amino acid sequence of SEQ ID NO:2: Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val, an N-terminal truncated fragment of the thrombin peptide derivative having at least fourteen amino acids, or a C-terminal truncated fragment of the thrombin peptide derivative comprising at least eighteen amino acids. X₁ is Glu or Gln and X₂ is Phe, Met, Leu, His or Val. In other specific embodiments, the thrombin peptide derivative is the polypeptide SEQ ID NO:3: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (TP508).

In further embodiments of the methods, the NPAR agonist is a modified thrombin peptide derivative disclosed herein. In specific embodiments, the modified thrombin peptide derivative comprises the amino acid sequence of SEQ ID NO:4: Arg-Gly-Asp-Ala-Xaa-X₁-Gly-Asp-Ser-Gly-Gly-Pro-X₂-Val, or a C-terminal truncated fragment thereof having at least six amino acids. In other specific embodiments, the modified thrombin peptide derivative comprises the amino acid sequence of SEQ ID NO:5: Ala-Gly-Tyr-Lys-Pro-Asp-Glu-Gly-Lys-Arg-Gly-Asp-Ala-Xaa-X_{I}-Gly-Asp-Ser-Gly-Gly-Pro-X₂-Val, or a fragment thereof comprising amino acids 10-18 of SEQ ID NO:5.

In still other embodiments, the NPAR agonist is a thrombin peptide derivative dimer of two thrombin peptide derivatives disclosed herein. More specifically, a thrombin peptide derivative dimer comprises in one instance the amino acid sequence Arg-Gly-Asp-Ala-Cys-X_{I}-Gly-Asp-Ser-Gly-Gly-Pro-X₂-Val (SEQ ID NO:1) or a C-terminal truncated fragment thereof having at least six amino acids. In other instances, the thrombin peptide derivative dimer comprises a polypeptide having the amino acid sequence of SEQ ID NO:2: Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val, or a fragment thereof comprising amino acids 10-18 of SEQ ID NO:2. In still other instances of the invention, the thrombin peptide derivative dimer is the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3). The thrombin peptide derivative dimer is represented by the structural formula (IV) in other instances.

In further embodiments, the NPAR agonist is an antibody or antigen-binding fragment thereof that binds to a complementary peptide, wherein the complementary peptide is encoded by the complement of a nucleotide sequence encoding a portion of thrombin.

The thrombin referred to above can be a mammalian thrombin, and in particular, a human thrombin. The portion of thrombin can be a thrombin receptor binding domain or a portion thereof. In one embodiment, the thrombin receptor binding domain or portion thereof comprises the amino acid sequence Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). Another portion of a thrombin receptor binding domain comprises the amino acid sequence Glu-Gly-Lys-Arg-Gly-Asp-Ala-Cys-Glu-Gly (SEQ ID NO:7).

The complementary peptide to which the antibody or the antigen-binding fragment thereof binds can be encoded by the 5′-3′ sequence of the antisense RNA strand or encoded by the 3′-5′ sequence of the antisense RNA strand.

In specific embodiments, the complementary peptide comprises the amino acid sequence Lys-Gly-Ser-Pro-Thr-Val-Thr-Phe-Thr-Gly-Ile-Pro-Cys-Phe-Pro-Phe-Ile-Arg-Leu-Val-Thr-Ser (SEQ ID NO:8) or Thr-Phe-Thr-Gly-Ile-Pro-Ser-Phe-Pro-Phe (SEQ ID NO:9) or Arg-Pro-Met-Phe-Gly-Leu-Leu-Pro-Phe-Ala-Pro-Leu-Arg-Thr-Leu-Pro-Leu-Ser-Pro-Pro-Gly-Lys-Gln (SEQ ID NO:10) or Lys-Pro-Phe-Ala-Pro-Leu-Arg-Thr-Leu-Pro (SEQ ID NO:11).

The NPAR agonist to be used in the methods of the invention can be a polyclonal antibody, or a monoclonal antibody or antigen-binding fragment thereof. In particular embodiments, these are human antibodies. Monoclonal antibodies to be used as NPAR agonists in methods of therapy can be humanized antibodies, chimeric antibodies or antigen-binding fragments of any of the foregoing, which can include Fab fragments, Fab′ fragments, F(ab′)₂ fragments and Fv fragments.

### BRIEF DESCRIPTION OF THE DRAWINGS

FIG. 1 is a graph showing densitometric analysis of a Western blot of activated endothelial nitric oxide synthase (eNOS) in HCAE cells following treatments with TP508, VEGF or a combination thereof.

FIG. 2A is a diagram showing the experimental apparatus and design of experiments to measure migration of endothelial cells toward a chemoattractant.

FIG. 2B is a bar graph showing the effect of TP508 treatment on migration of endothelial cells toward the angiogenic factor VEGF.

FIG. 3A is a diagram showing the experimental apparatus and design of experiments to measure invasion of endothelial cells through Matrigel toward a chemoattractant.

FIG. 3B is a bar graph showing the effect of TP508 treatment on invasion of endothelial cells toward the angiogenic factor VEGF.

FIG. 4 depicts the encoded amino acid sequence of human pro-thrombin (SEQ ID NO:12). Amino acids 508-530, which contain the thrombin receptor binding domain, are underlined. Thrombin consists of the C-terminal 579 amino acid residues of prothrombin. See GenBank Accession No. AJ972449.

FIG. 5A is a diagram of the apparatus and design of the assay used to test the invasion of human coronary artery endothelial (HCAE) cells through a matrix in response to basic fibroblast growth factor (bFGF), as described in Example 4.

FIG. 5B is a bar graph showing the extent of invasion of HCAE cells in response to medium containing bFGF (FGF), or in response to medium without bFGF (CTR) as described in Example 4.

FIG. 6A is a diagram of the apparatus and design of the assay used to test the migration of HCAE cells through a fibronectin insert in response to bFGF, as described in Example 4.

FIG. 6B is a bar graph showing the extent of migration of HCAE cells in response to medium containing bFGF (FGF), or in response to medium without bFGF (CTR), as described in Example 4.

FIG. 7A is a diagram of the apparatus and design of the assay used to test the invasion of human coronary artery endothelial (HCAE) cells through a matrix in response to platelet derived growth factor (PDGF), as described in Example 5.

FIG. 7B is a bar graph showing the extent of invasion of HCAE cells in response to medium containing PDGF (PDGF), or in response to medium without PDGF (CTR), as described in Example 5.

FIG. 8A is a diagram of the apparatus and design of the assay used to test the migration of HCAE cells through a fibronectin insert in response to PDGF, as described in Example 5.

FIG. 8B is a bar graph showing the extent of migration of HCAE cells in response to medium containing PDGF (PDGF), or in response to medium without PDGF (CTR), as described in Example 5.

### DETAILED DESCRIPTION OF THE INVENTION

It is demonstrated herein, using a system to measure the response of human coronary artery endothelial cells to angiogenic factors, that TP508 treatment more than doubles the angiogenic potential of VEGF for endothelial cells, both under normoxic and hypoxic conditions. TP508 [the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3)] restores the ability of VEGF to activate eNOS (endothelial nitric oxide synthase), thereby increasing NO required to induce angiogenesis.

The invention encompasses methods of combination therapy, wherein an angiogenic factor and an NPAR agonist are both administered in an amount and for a duration effective to promote cardiac tissue repair, cardiac revascularization, vascular endothelial cell proliferation and/or endothelial cell migration in a subject in need of treatment for one or more of these. The combination is a combination of angiogenic factor and NPAR agonist.

Previously, the NPAR agonist TP508 was shown to stimulate proliferation and migration of endothelial cells and to stimulate angiogenesis when administered alone. Further, treatment with TP508 alone was effective in restoring the function of ischemic heart muscle and in stimulating myocardial revascularization. See U.S. Pat. No. 6,861,407, the contents of which are herein incorporated by reference in their entirety.

The invention encompasses methods of promoting cardiac tissue repair, methods of promoting cardiac revascularization, methods of promoting vascular endothelial cell proliferation, and methods of promoting vascular endothelial cell migration. The method, in each case, includes administering to the subject to be treated, a combination in a therapeutically effective amount, the combination comprising one or more angiogenic growth factors, and one or more agonists of the non-proteolytically activated thrombin receptor (NPAR agonists). In particular embodiments, the invention encompasses the above methods of promoting cardiac tissue repair, cardiac revascularization, vascular endothelial cell proliferation, and vascular endothelial cell migration, using any of VEGF, bFGF or platelet derived growth factor (PDGF) in combination with an NPAR agonist such as TP508.

The invention also encompasses methods of promoting cardiac tissue repair, methods of promoting cardiac revascularization, methods of promoting vascular endothelial cell proliferation, and methods of promoting vascular endothelial cell migration, wherein the method comprises administering to the subject a combination in a therapeutically effective amount, the combination consisting essentially of an angiogenic growth factor and an agonist of the non-proteolytically activated thrombin receptor. In this method an angiogenic growth factor and an agonist of the non-proteolytically activated thrombin receptor (NPAR agonist) are administered in combination as the only therapeutically active agents. In particular embodiments, the invention encompasses the above methods of promoting cardiac tissue repair, cardiac revascularization, vascular endothelial cell proliferation, and vascular endothelial cell migration, using any of VEGF, bFGF or platelet derived growth factor (PDGF) in combination with an NPAR agonist such as TP508.

The angiogenic growth factors in the methods can be any of the angiogenic growth factors known to those of skill in the art, for example, those listed in Tables 1 and 2. Preferred angiogenic growth factors are human. In some embodiments, the angiogenic growth factors are those of the VEGF family. In other embodiments, the angiogenic growth factor is human VEGF-A.

In some embodiments of the described methods, the NPAR agonist is a thrombin peptide derivative disclosed herein. More specifically, one thrombin peptide derivative comprises the amino acid sequence of Arg-Gly-Asp-Ala-Cys-X₁-Gly-Asp-Ser-Gly-Gly-Pro-X₂-Val (SEQ ID NO:1), or a C-terminal truncated fragment thereof comprising at least six amino acids. In specific embodiments, the thrombin peptide derivative comprises the amino acid sequence of SEQ ID NO:2: Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val, an N-terminal truncated fragment of the thrombin peptide derivative having at least fourteen amino acids, or a C-terminal truncated fragment of the thrombin peptide derivative comprising at least eighteen amino acids. X₁ is Glu or Gln and X₂ is Phe, Met, Leu, His or Val. In other specific embodiments, the thrombin peptide derivative is the polypeptide SEQ ID NO:3: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (TP508).

In further embodiments of the methods, the NPAR agonist is a modified thrombin peptide derivative disclosed herein. In specific embodiments, the modified thrombin peptide derivative comprises the amino acid sequence of SEQ ID NO:4: Arg-Gly-Asp-Ala-Xaa-X₁-Gly-Asp-Ser-Gly-Gly-Pro-X₂-Val, or a C-terminal truncated fragment thereof having at least six amino acids. In other specific embodiments, the modified thrombin peptide derivative comprises the amino acid sequence of SEQ ID NO:5: Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val, or a fragment thereof comprising amino acids 10-18 of SEQ ID NO:5.

In still other embodiments, the NPAR agonist is a thrombin peptide derivative dimer of two thrombin peptide derivatives disclosed herein. More specifically, a thrombin peptide derivative dimer comprises in one instance the amino acid sequence Arg-Gly-Asp-Ala-Cys-X₁-Gly-Asp-Ser-Gly-Gly-Pro-X₂-Val (SEQ ID NO:1) or a C-terminal truncated fragment thereof having at least six amino acids. In other instances, the thrombin peptide derivative dimer comprises a polypeptide having the amino acid sequence of SEQ ID NO:2: Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val, or a fragment thereof comprising amino acids 10-18 of SEQ ID NO:2. In still other instances of the invention, the thrombin peptide derivative dimer is the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3). The thrombin peptide derivative dimer is represented by the structural formula (IV) in other instances.

In further embodiments, the NPAR agonist is an antibody or antigen-binding fragment thereof that binds to a complementary peptide, wherein the complementary peptide is encoded by the complement of a nucleotide sequence encoding a portion of thrombin.

The thrombin referred to above can be a mammalian thrombin, and in particular, a human thrombin. The portion of thrombin can be a thrombin receptor binding domain or a portion thereof. In one embodiment, the thrombin receptor binding domain or portion thereof comprises the amino acid sequence Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). Another portion of a thrombin receptor binding domain comprises the amino acid sequence Glu-Gly-Lys-Arg-Gly-Asp-Ala-Cys-Glu-Gly (SEQ ID NO:7).

The complementary peptide to which the antibody or the antigen-binding fragment thereof binds can be encoded by the 5′-3′ sequence of the antisense RNA strand or encoded by the 3′-5′ sequence of the antisense RNA strand.

In specific embodiments, the complementary peptide comprises the amino acid sequence Lys-Gly-Ser-Pro-Thr-Val-Thr-Phe-Thr-Gly-Ile-Pro-Cys-Phe-Pro-Phe-Ile-Arg-Leu-Val-Thr-Ser (SEQ ID NO:8) or Thr-Phe-Thr-Gly-Ile-Pro-Ser-Phe-Pro-Phe (SEQ ID NO:9) or Arg-Pro-Met-Phe-Gly-Leu-Leu-Pro-Phe-Ala-Pro-Leu-Arg-Thr-Leu-Pro-Leu-Ser-Pro-Pro-Gly-Lys-Gln (SEQ ID NO:10) or Lys-Pro-Phe-Ala-Pro-Leu-Arg-Thr-Leu-Pro (SEQ ID NO:11).

The NPAR agonist to be used in the methods of the invention can be a polyclonal antibody, or a monoclonal antibody or antigen-binding fragment thereof. In particular embodiments, these are human antibodies. Monoclonal antibodies to be used as NPAR agonists in methods of therapy can be humanized antibodies, chimeric antibodies or antigen-binding fragments of any of the foregoing, which can include Fab fragments, Fab′ fragments, F(ab′)₂ fragments and Fv fragments.

Compounds which stimulate NPAR are said to be NPAR agonists. NPAR is a high-affinity thrombin receptor present on the surface of most cells. This NPAR component is largely responsible for high-affinity binding of thrombin, proteolytically inactivated thrombin, and thrombin derived peptides to cells. NPAR appears to mediate a number of cellular signals that are initiated by thrombin independent of its proteolytic activity. An example of one such signal is the upregulation of annexin V and other molecules identified by subtractive hybridization (see Sower, et. al., Experimental Cell Research 247:422 (1999)). NPAR is therefore characterized by its high affinity interaction with thrombin at cell surfaces and its activation by proteolytically inactive derivatives of thrombin and thrombin derived peptide agonists as described below. NPAR activation can be assayed based on the ability of molecules to stimulate cell proliferation when added to fibroblasts in the presence of submitogenic concentrations of thrombin or molecules that activate protein kinase C, as disclosed in U.S. Pat. Nos. 5,352,664 and 5,500,412. The entire teachings of these patents are incorporated herein by reference. NPAR agonists can be identified by this activation or by their ability to compete with ¹²⁵I-thrombin binding to cells.

A thrombin receptor binding domain is defined as a polypeptide or portion of a polypeptide which directly binds to the thrombin receptor and/or competitively inhibits binding between high-affinity thrombin receptors and alpha-thrombin.

NPAR agonists to be used in any of the methods described herein include thrombin derivative peptides, modified thrombin peptide derivatives, thrombin derivative peptide dimers and NPAR agonist antibodies to complementary peptides of thrombin as disclosed herein.

### Angiogenic Growth Factors

An “angiogenic growth factor” is a polypeptide which stimulates the development of blood vessels, e.g., promotes angiogenesis, endothelial cell growth, stability of blood vessels, and/or vasculogenesis. For example, angiogenic factors, include, but are not limited to, e.g., VEGF and members of the VEGF family, PlGF, PDGF family, fibroblast growth factor family (FGFs), TIE ligands (Angiopoietins), ephrins, ANGPTL3, ANGPTL4, etc. Angiogenic factors also include polypeptides such as growth hormone, insulin-like growth factor-I (IGF-I), VIGF, epidermal growth factor (EGF), CTGF and members of its family, and TGF-α and TGF-β. See, e.g., Klagsbrun and D'Amore, Annu. Rev. Physiol., 53:217-39 (1991); Streit and Detmar, Oncogene, 22:3172-3179 (2003); Ferrara & Alitalo, Nature Medicine 5(12):1359-1364 (1999); Tonini et al., Oncogene, 22:6549-6556 (2003); and, Sato Int. J. Clin. Oncol., 8:200-206 (2003).

The term “VEGF” (also referred to as “VEGF-A”) as used herein refers to vascular endothelial cell growth factor protein A. The term “human VEGF” (also referred to as “human VEGF-A”) as used herein refers to any of the isoforms of human vascular endothelial cell growth factor. Described isoforms (arising by differential mRNA splicing) include 121, 145, 148, 165, 165b, 183, 189 and 206. See, for example, Table 1 and Leung et al., Science 246:1306 (1989), and Houck et al., Mol. Endocrin. 5:1806 (1991). “Human VEGF” also includes naturally occurring allelic variants of human VEGF-A and variants arising by variations in post-translational modifications. Table 1 is not intended to be comprehensive or limiting. Angiogenic growth factors in Table 1 are human unless otherwise indicated.

TABLE 1
Examples of Angiogenic Growth Factors of VEGF Family

| Angiogenic Growth | | | Chromosome | GenBank | |
|---|---|---|---|---|---|
| Factor | | Receptor | Location | No. | Reference |
| Human VEGF-A | | VEGFR1 | 6p12 | NM_003376 | |
| | | VEGFR2 | | | |
| isoforms: | 121 | | | | |
| | 145 | | | | |
| | 148 | | | | |
| | 165 | | | | |
| | 165b | | | | |
| | 183 | | | | |
| | 189 | | | | |
| | 206 | | | | |
| Human VEGF-B | | VEGFR1 | 11q13 | NM_003377 | |
| Human VEGF-C | | VEGFR3 | 4q34.1- | NM_005429 | |
| | | VEGFR2 | q34.3 | | |
| Human VEGF-D | | VEGFR3 | Xp22.31 | NM_004469 | |
| | | VEGFR2 | | | |
| VEGF-E [Orf virus | | VEGFR2 | | AF106020 | |
| (D1701)] | | | | | |
| VEGF-E [Orf virus | | VEGFR2 | | S67520 | |
| (NZ2)] | | | | | |
| VEGF-E [Orf virus | | VEGFR2 | | S67522 | |
| (NZ7)] | | | | | |
| VEGF-E_{N27}/PlGF | | VEGFR2 | | | 1, 2 |
| (chimeric) | | | | | |
| VEGF-E/PlGF | | VEGFR2 | | | 3 |
| (chimeric) | | | | | |
| PlGF | | FLT1 | 14q22- | NM_002632 | |
| | | VEGFR1 | q24.3 | | |
| isoforms: | PlGF1 | | | | |
| | PlGF2 | | | | |
| | PlGF3 | | | | |
| | PlGF4 | | | | |

| | | | |
|---|---|---|---|
| VEGF-F (viper) | VEGFR2 | | 4 |
| D. melanogaster | | NM_078683 | 5 |
| PVF1 | | | |
| D. melanogaster | | NM_078775 | 5 |
| PVF2 | | | |
| D. melanogaster | | NM_078776 | 5 |
| PVF3 | | | |

### REFERENCES

1. Zheng, Y. et al., “Chimeric VEGF-ENZ7/PlGF Promotes Angiogenesis Via VEGFR-2 Without Significant Enhancement of Vascular Permeability and Inflammation,” Arterioscler. Throm. Vasc. Biol. 26: 2019-2026 (2006).
2. Zheng, Y. et al., “Chimeric VEGF-ENZ7/PlGF Specifically Binding to VEGFR-2 Accelerates Skin Wound Healing via Enhancement of Neovascularization,” Arterioscler. Throm. Vasc. Biol. 27: 503-511 (2007).
3. Inoue, N. et al., “Therapeutic Angiogenesis Using Novel Vascular Endothelial Growth Factor-E/Human Placental Growth Factor Chimera Genes,” Arterioscler. Throm. Vasc. Biol. 27: 99-105 (2007).
4. Suto, K. et al., “Crystal structures of novel vascular endothelial growth factors (VEGF) from snake venoms: insight into selective VEGF binding to kinase insert domain-containing receptor but not to fms-like tyrosine kinase-1,” J. Biol. Chem. 280(3): 2126-2131 (2005).
5. Duchek, P., “Guidance of cell migration by the Drosophila PDGF/VEGF receptor,” Cell 107(1): 17-26 (2001).

Human VEGF-A exists as a number of isoforms that arise from alternative splicing of mRNA of a single gene organized into 8 exons located on chromosome 6 (see, e.g., Ferrara N, Davis Smyth T. Endocr Rev 18:1-22 (1997); and, Henry and Abraham, Review of Preclinical and Clinical Results with Vascular Endothelial Growth Factors for Therapeutic Angiogenesis, Current Interventional Cardiology Reports, 2:228-241 (2000)). See also, U.S. Pat. Nos. 5,332,671 and 6,899,882. In one embodiment, VEGF₁₆₅ is administered in the methods of the invention (e.g., recombinant human VEGF₁₆₅). VEGF₁₆₅, the most abundant isoform, is a basic, heparin binding, dimeric covalent glycoprotein with a molecular mass of about 45,000 Daltons (Id). VEGF₁₆₅ homodimer consists of two 165 amino acid chains. The protein has two distinct domains: a receptor binding domain (residues 1-110) and a heparin binding domain (residues 110-165). The domains are stabilized by seven intramolecular disulfide bonds, and the monomers are linked by two interchain disulfide bonds to form the native homodimer. VEGF₁₂₁ lacks the heparin binding domain (see, e.g., U.S. Pat. No. 5,194,596), whereas VEGF₁₈₉ (see, e.g., U.S. Pat. Nos. 5,008,196; 5,036,003; and 5,240,848) and VEGF₂₀₆ are sequestered in the extracellular matrix.

The term “angiogenic growth factor” also includes those below in Table 2. The list in Table 2 is not intended to be comprehensive or limiting.

TABLE 2

Examples of Other Angiogenic Growth Factors
Angiogenin
Angiopoietin-1
Del-1
Acidic fibroblast growth factor (aFGF or FGF-1^{1, 2})
Basic fibroblast growth factor (bFGF or FGF-2^{3, 4})
Fibroblast growth factor 4 (FGF 4)
Follistatin
Granulocyte colony-stimulating factor (G-CSF)
Hepatocyte growth factor (HGF)/scatter factor (SF)
Interleukin-8 (IL-8)
Leptin
Midkine
Platelet-derived endothelial cell growth factor (PD-ECGF)
Platelet-derived growth factor-BB (PDGF-BB)
(rhPDGF-BB is Regranex ®)
Pleiotrophin (PTN)
Progranulin
Proliferin
Transforming growth factor-alpha (TGF-alpha)
Transforming growth factor-beta (TGF-beta)
Tumor necrosis factor-alpha (TNF-alpha)
Vascular endothelial growth factor (VEGF)/
vascular permeability factor (VPF)
Thymosin beta 4 (Tβ4)
Connective tissue growth factor
Osteopontin
Insulin growth factor (IGF-1)

| Angiogenic Growth | | Chromosome | GenBank | |
|---|---|---|---|---|
| Factor | Receptor | Location | No. | Reference |
| PDGFD | PDGFR-α | 11q22.3 | NM_025208 | |
| | PDGFR-β | | | |
| PDGF-α | | 7p22 | NM_002607 | |
| PDGF2 | | 22q12.3- | NM_002608 | |
| | | q13.1 | | |
| isoforms: | | | | |
| 1 (241 | | | | |
| amino acids) | | | | |
| 2 (226 | | | | |
| amino acids) | | | | |
| PDGFC | PDGF-α | 4q32 | NM_016205 | |

### REFERENCES

1. Schumacher, B., et al., “Induction of neoangeogenesis in ischemic myocardium by human growth factors: first clinical results of a new treatment of coronary heart disease,” Circulation 97:645-650 (1998).
2. Stegmann, T. J., et al., “Induction of myocardial neoangiogenesis by human growth factors: a new therapeutic option in coronary heart disease,” Herz 25:589-599 (2000).
3. Selke, F. W., et al., “Therapeutic angiogenesis with basic fibroblast growth factor: technique and early results,” Ann. Thorac. Surg. 65:1540-1544 (1998).
4. Laham, R. J., et al., “Local perivascular delivery of basic fibroblast growth factor in patients undergoing coronary bypass surgery: results of a phase 1 randomized, double-blind, placebo-controlled trial,” Circulation 100:1865-1871 (1999).

A “native” polypeptide (e.g., a native angiogenic factor) is a polypeptide having the same amino acid sequence as a polypeptide isolated from a natural source. Thus, a native polypeptide can have the amino acid sequence of naturally occurring polypeptide from any mammal, e.g., a human. Such native polypeptides can be isolated from nature or can be produced by recombinant or synthetic means. The term native polypeptide encompasses naturally occurring truncated or secreted forms of the polypeptide (e.g., an extracellular domain sequence), allelic forms designated as wild type, naturally occurring variant forms (e.g., alternatively spliced iso forms) and naturally occurring allelic variants of the polypeptide.

A “polypeptide variant” (e.g., a polypeptide variant of an angiogenic factor) means a biologically active polypeptide having at least about 80% amino acid sequence identity with the native polypeptide. Such “polypeptide variants” include, for instance, polypeptides wherein one or more amino acid residues are added, or deleted, at the N- and/or C-terminus of the polypeptide relative to a native polypeptide. Ordinarily, a polypeptide variant will have at least about 80% amino acid sequence identity, or at least about 90% amino acid sequence identity, or at least about 95% or more amino acid sequence identity with the native polypeptide. Polypeptide variants include polypeptides that comprise one or more amino acid substitutions, additions or deletions., or combinations of any of these differences from the native polypeptide. Polypeptide variants can have, for instance, several, such as 5 to 10, 1 to 5, or 4, 3, 2 or 1 amino acids substituted, deleted, or added, in any combination, compared to native polypeptides. In one embodiment, variants have silent substitutions, additions and/or deletions that do not significantly alter the properties and activities of the polypeptide compared to the native polypeptide. Polypeptide variants can also be modified polypeptides in which one or more amino acid residues are modified. Polypeptide variants can be prepared by a variety of methods well known in the art. Polypeptide variants differing by amino acid sequence from a native polypeptide can be prepared by mutations in the encoding DNA. Polypeptide variants also include polypeptides that differ from native polypeptides in glycosylation or other post-translational modification.

A polypeptide variant can be prepared, for instance, by site-directed mutagenesis of nucleotides in the DNA encoding the native polypeptide or by phage display techniques, thereby producing DNA encoding the variant, and thereafter expressing the DNA in recombinant cell culture.

Amino acid deletions generally range from about 1 to 30 residues, optionally 1 to 10 residues, optionally 1 to 5 or less, and typically are contiguous.

Amino acid sequence additions include amino- and/or carboxyl-terminal fusions of from one residue to polypeptides of essentially unrestricted length as well as intrasequence insertions of single or multiple amino acid residues. Intrasequence additions (i.e., additions within a native polypeptide sequence) may range generally from about 1 to 10 residues, optionally 1 to 5, or optionally 1 to 3. An example of a terminal insertion includes a fusion of a signal sequence, whether heterologous or homologous to the host cell, to the N-terminus to facilitate the secretion from recombinant hosts.

Additional polypeptide variants are those in which at least one amino acid residue in the native polypeptide has been removed and a different amino acid residue inserted in its place (substitution). Conservative substitutions in polypeptide variants of an angiogenic growth factor may be made in accordance with those shown in Table 3, wherein both exemplary and preferred substitutions are conservative substitutions in polypeptide variants of an angiogenic growth factor. Polypeptide variants can also comprise unnatural amino acids as described herein.

Amino acids may be grouped according to similarities in the properties of their side chains (A. L. Lehninger, Biochemistry, second ed., pp. 73-75, Worth Publishers, New York (1975)):

(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (O)
(3) acidic: Asp (D), Glu (E)
(4) basic: Lys (K), Arg (R), His(H)

Alternatively, naturally occurring amino acids may be divided into groups based on common side-chain properties:

(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

TABLE 3

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

“Naturally occurring amino acid residues” (i.e. amino acid residues encoded by the genetic code) may be selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). A “non-naturally occurring amino acid residue” refers to an amino acid residue, other than those naturally occurring amino acid residues listed above, which can be bound to adjacent amino acid residues(s) in a polypeptide chain through peptide bonds. Examples of non-naturally occurring amino acid residues include, e.g., norleucine, ornithine, norvaline, homoserine and other amino acid residue analogues such as those described in Ellman et al. Meth. Enzym. 202:301-336 (1991) and US Patent application publications 20030108885 and 20030082575.

“Percent (%) amino acid sequence identity” herein is defined as the percentage of amino acid residues in a candidate sequence of an angiogenic growth factor that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full length of the sequences being compared.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Portions of an angiogenic growth factor include polypeptides that are shorter than a corresponding native polypeptide, and comprise at least 20 contiguous amino acid residues of the corresponding native polypeptide, that share 75% to 100% amino-acid sequence identity with the native polypeptide. In particular embodiments, the portion shares at least 90% or 95% amino acid sequence identity with the native polypeptide. Portions of an angiogenic growth factor can be synthesized, and can have an N-terminal amino group and a C-terminal carboxyl group as they occur in proteins isolated from natural sources, or can have a modified N-terminus (e.g., acylated) and/or a modified C-terminus (e.g., amidated). Portions of an angiogenic growth factor can be generated through the expression of genes constructed for the purpose of producing the portion. Portions may be cyclic or linear. In all cases, portions of an angiogenic growth factor have at least 50% of the biological activity of the corresponding native polypeptide, as measured by an assay appropriate to measuring the angiogenic activity of the corresponding native polypeptide.

A number of assays have been used previously to measure angiogenic activity and have been described. An angiogenic growth factor, whether it is a native polypeptide, polypeptide variant, portion of an angiogenic growth factor, or fusion protein of an angiogenic growth factor, can be tested by an in vitro or in vivo assay to assess its activity, using one or more of the assays described herein, or other suitable assay such as those known to persons of ordinary skill in the art. Not all assays are appropriate to measure the angiogenic activity of a given angiogenic growth factor.

A rabbit corneal assay has been described, in which angiogenic growth factor implanted into cornea stimulates the growth of new capillaries. See Ziche et al., Lab. Invest. 61:629-634 (1989). An in vitro angiogenesis assay system allows for observation of morphological changes in endothelial cells stimulated by angiogenic growth factor. See Montesano et al., J. Cell Biol. 97:1648-1652 (1983). Angiogenic growth activity can also be measured by an assay for cell growth [Marconcini et al., Proc. Natl. Acad. Sci. USA 96: 9671-9676 (1999)] in response to the angiogenic growth factor, or by invasion and migration assays such as those described in Examples 2-5.

Endothelial cells are activated by and migrate toward angiogenic factors. In the early stages of the angiogenesis process, the activated endothelial cells express significant levels of matrix degrading enzymes, matrix metalloproteinases (MMPs), that digest the capillary basement membrane and allow the cells to move toward an angiogenic stimulus. Invasion and migration assays are in vitro techniques designed to investigate this process. Migration (or chemotaxis) is the directional movement of cells in response to a concentration gradient of a soluble attractant. Invasion differs from migration in that, in addition to the migratory response, the cells must express significant quantities of MMPs that degrade the matrix barrier. This allows cell movement into and through the extracellular matrix.

A fusion protein of an angiogenic growth factor comprises a biologically active native polypeptide or biologically active portion thereof (as described above) as a first moiety, linked to second moiety not occurring in the native polypeptide. Thus, the second moiety can be an amino acid or polypeptide. The first moiety can be in an N-terminal location, C-terminal location or internal to the fusion protein. In one embodiment, the fusion protein comprises a biologically active polypeptide that consists of the amino acid sequence of a naturally occurring angiogenic growth factor or biologically active portion thereof as the first moiety, and a second moiety comprising a linker sequence and an affinity ligand.

A fusion protein of an angiogenic growth factor can be produced by a variety of methods. For example, a fusion protein can be produced by the insertion of gene encoding an angiogenic growth factor or portion thereof into a suitable expression vector. The resulting construct can be introduced into a suitable host cell for expression. Upon expression, fusion protein can be purified from a cell lysate by means of a suitable affinity matrix, for example (see e.g., Current Protocols in Molecular Biology. Ausubel, F. M. et al., eds., pp. 16.4.1-16.7.8, containing supplements up through Supplement 28, 1994). See, for examples of VEGF fusion (chimeric) proteins, Zheng, et al., Arterioscler. Thromb. Vas. Biol. 2006; 26: 2019-2026 and Inoue, et al., Arterioscler. Thromb. Vasc. Biol. 2007; 27: 99-105.

Angiogenic growth factors can be of human origin or of non-human (preferably mammalian) origin. Human angiogenic growth factors as well as non-human species homologs are angiogenic growth factors and can be used in the combination therapies of the invention. A homolog preferably has at least 70% amino acid sequence identity, more preferably, at least 80% sequence identity and, even more preferably, at least 90% sequence identity with a human angiogenic growth factor.

In accordance with the present invention, “angiogenic growth factors” encompass native angiogenic growth factors, portions of angiogenic growth factors, polypeptide variants of angiogenic growth factors and fusion proteins of angiogenic growth factors as described above.

### NPAR Agonists

Compounds which stimulate NPAR are said to be NPAR agonists. NPAR is a high-affinity thrombin receptor present on the surface of most cells. This NPAR component is largely responsible for high-affinity binding of thrombin, proteolytically inactivated thrombin, and thrombin derived peptides to cells. NPAR appears to mediate a number of cellular signals that are initiated by thrombin independent of its proteolytic activity. An example of one such signal is the upregulation of annexin V and other molecules identified by subtractive hybridization (see Sower, et. al., Experimental Cell Research 247:422 (1999)). NPAR is therefore characterized by its high affinity interaction with thrombin at cell surfaces and its activation by proteolytically inactive derivatives of thrombin and thrombin derived peptide agonists as described below. NPAR activation can be assayed based on the ability of molecules to stimulate cell proliferation when added to fibroblasts in the presence of submitogenic concentrations of thrombin or molecules that activate protein kinase C, as disclosed in U.S. Pat. Nos. 5,352,664 and 5,500,412. The entire teachings of these patents are incorporated herein by reference. NPAR agonists can be identified by this activation or by their ability to compete with ¹²⁵I-thrombin binding to cells.

A thrombin receptor binding domain is defined as a polypeptide or portion of a polypeptide which directly binds to the thrombin receptor and/or competitively inhibits binding between high-affinity thrombin receptors and alpha-thrombin. NPAR agonists of the present invention include thrombin derivative peptides, modified thrombin derivative peptides, thrombin derivative peptide dimers and NPAR agonist antibodies to complementary peptides of thrombin as disclosed herein.

### Thrombin Derivative Peptides

Among NPAR agonists are thrombin peptide derivatives (also: “thrombin derivative peptides”), which are analogs of thrombin that have an amino acid sequence derived at least in part from that of thrombin and are active at the non-proteolytically activated thrombin receptor. Thrombin peptide derivatives include, for example, peptides that are produced by recombinant DNA methods, peptides produced by enzymatic digestion of thrombin, and peptides produced synthetically, which can comprise amino acid substitutions compared to thrombin and/or modified amino acids, especially at the termini.

NPAR agonists of the present invention include thrombin derivative peptides described in U.S. Pat. Nos. 5,352,664 and 5,500,412. In one embodiment, the NPAR agonist of the present invention is a thrombin peptide derivative or a physiologically functional equivalent, i.e., a polypeptide with no more than about fifty amino acids, preferably no more than about thirty amino acids and having sufficient homology to the fragment of human thrombin corresponding to thrombin amino acids 508-530 (Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val; SEQ ID NO:6) that the polypeptide activates NPAR. In one instance, the thrombin peptide derivative is a 23-amino acid polypeptide comprising the amino acid sequence SEQ ID NO:6. The thrombin peptide derivatives or modified thrombin peptide derivatives described herein preferably have from about 12 to about 23 amino acid residues, more preferably from about 19 to about 23 amino acid residues.

In another embodiment, the NPAR agonist of the present invention is a thrombin peptide derivative comprising a moiety represented by Structural Formula (I):

Asp-Ala-R (I).

R is a serine esterase conserved domain. Serine esterases, e.g., trypsin, thrombin, chymotrypsin and the like, have a region that is highly conserved. “Serine esterase conserved domain” refers to a polypeptide having the amino acid sequence of one of these conserved regions or is sufficiently homologous to one of these conserved regions such that the thrombin peptide derivative retains NPAR activating ability.

A physiologically functional equivalent of a thrombin derivative encompasses molecules which differ from thrombin derivatives in particulars which do not affect the function of the thrombin receptor binding domain or the serine esterase conserved amino acid sequence. Such particulars may include, but are not limited to, conservative amino acid substitutions as defined below for NPAR agonists, and modifications, for example, amidation of the carboxyl terminus, acylation (e.g. acetylation) of the amino terminus, conjugation of the polypeptide to a physiologically inert carrier molecule, or sequence alterations in accordance with the serine esterase conserved sequences.

A domain having a serine esterase conserved sequence can comprise a polypeptide sequence containing at least 4-12 of the N-terminal amino acids of the dodecapeptide previously shown to be highly conserved among serine proteases (Asp-X₁-Cys-X₂-Gly-Asp-Ser-Gly-Gly-Pro-X₃-Val; SEQ ID NO:13); wherein X₁ is either Ala or Ser; X₂ is either Glu or Gln; and X₃ is Phe, Met, Leu, His, or Val).

In one embodiment, the serine esterase conserved sequence comprises the amino acid sequence of SEQ ID NO:14 (Cys-Glu-Gly-Asp-Ser-Gly-Gly-Pro-Phe-Val) or a C-terminal truncated fragment of a polypeptide having the amino acid sequence of SEQ ID NO: 14. It is understood, however, that zero, one, two or three amino acids in the serine esterase conserved sequence can differ from the corresponding amino acid in SEQ ID NO:14. Preferably, the amino acids in the serine esterase conserved sequence which differ from the corresponding amino acid in SEQ ID NO:14 are conservative substitutions as defined below for NPAR agonists, and are more preferably highly conservative substitutions. A “C-terminal truncated fragment” refers to a fragment remaining after removing an amino acid or block of amino acids from the C-terminus, said fragment having at least six and more preferably at least nine amino acids.

In another embodiment, the serine esterase conserved sequence comprises the amino acid sequence of SEQ ID NO:15 (Cys-X₁-Gly-Asp-Ser-Gly-Gly-Pro-X₂-Val; X₁ is Glu or Gln and X₂ is Phe, Met, Leu, His or Val) or a C-terminal truncated fragment thereof having at least six amino acids, preferably at least nine amino acids.

In a preferred embodiment, the thrombin peptide derivative comprises a serine esterase conserved sequence and a polypeptide having a more specific thrombin amino acid sequence Arg-Gly-Asp-Ala (SEQ ID NO:16). One example of a thrombin peptide derivative of this type comprises Arg-Gly-Asp-Ala-Cys-X₁-Gly-Asp-Ser-Gly-Gly-Pro-X₂-Val (SEQ ID NO:1). X₁ and X₂ are as defined above. The thrombin peptide derivative can comprise the amino acid sequence of SEQ ID NO:6 (Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val) or an N-terminal truncated fragment thereof, provided that zero, one, two or three amino acids at positions 1-9 in the thrombin peptide derivative differ from the amino acid at the corresponding position of SEQ ID NO:6. Preferably, the amino acid residues in the thrombin peptide derivative which differ from the corresponding amino acid residues in SEQ ID NO:6 are conservative substitutions as defined below for NPAR agonists (thrombin peptide derivatives), and are more preferably highly conservative substitutions. An “N-terminal truncated fragment” refers to a fragment remaining after removing an amino acid or block of amino acids from the N-terminus, preferably a block of no more than six amino acids, more preferably a block of no more than three amino acids.

Optionally, the thrombin peptide derivatives described herein can be amidated at the C-terminus and/or acylated at the N-terminus. In a specific embodiment, the thrombin peptide derivatives comprise a C-terminal amide and optionally comprise an acylated N-terminus, wherein said C-terminal amide is represented by —C(O)NRₐR_{b}, wherein Rₐ and R_{b} are independently hydrogen, a C₁-C₁₀ substituted or unsubstituted aliphatic group, or Rₐ and R_{b}, taken together with the nitrogen to which they are bonded, form a C₁-C₁₀ non-aromatic heterocyclic group, and said N-terminal acyl group is represented by R_{c}C(O)—, wherein R_{c} is hydrogen, a C₁-C₁₀ substituted or unsubstituted aliphatic group, or a C₁-C₁₀ substituted or unsubstituted aromatic group. In another specific embodiment, the N-terminus of the thrombin peptide derivative is free (i.e., unsubstituted) and the C-terminus is free (i.e., unsubstituted) or amidated, preferably as a carboxamide (i.e., —C(O)NH₂). In a specific embodiment, the thrombin peptide derivative comprises the following amino acid sequence: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). In another specific embodiment, the thrombin peptide derivative comprises the amino sequence of Arg-Gly-Asp-Ala-Cys-Glu-Gly-Asp-Ser-Gly-Gly-Pro-Phe-Val (SEQ ID NO:17). Alternatively, the thrombin peptide derivative comprises the amino acid sequence of SEQ ID NO:18: Asp-Asn-Met-Phe-Cys-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe. The thrombin peptide derivatives comprising the amino acids of SEQ ID NO: 6, 17, or 18 can optionally be amidated at the C-terminus and/or acylated at the N-terminus. Preferably, the N-terminus is free (i.e., unsubstituted) and the C-terminus is free (i.e., unsubstituted) or amidated, preferably a carboxamide (i.e., —C(O)NH₂). It is understood, however, that zero, one, two or three amino acids at positions 1-9 and 14-23 in the thrombin peptide derivative can differ from the corresponding amino acid in SEQ ID NO:6. It is also understood that zero, one, two or three amino acids at positions 1-14 and 19-33 in the thrombin peptide derivative can differ from the corresponding amino acid in SEQ ID NO:18. Preferably, the amino acids in the thrombin peptide derivative which differ from the corresponding amino acid in SEQ ID NO:6 or SEQ ID NO:18 are conservative substitutions as defined below, and are more preferably highly conservative substitutions. Alternatively, an N-terminal truncated fragment of the thrombin peptide derivative having at least fourteen amino acids or a C-terminal truncated fragment of the thrombin peptide derivative having at least eighteen amino acids is a thrombin peptide derivative to be used as an NPAR agonist.

A “C-terminal truncated fragment” refers to a fragment remaining after removing an amino acid or block of amino acids from the C-terminus. An “N-terminal truncated fragment” refers to a fragment remaining after removing an amino acid or block of amino acids from the N-terminus. It is to be understood that the terms “C-terminal truncated fragment” and “N-terminal truncated fragment” encompass acylation at the N-terminus and/or amidation at the C-terminus, as described above.

A preferred thrombin peptide derivative for use in the disclosed method comprises the amino acid sequence SEQ ID NO:2: Ala-Gly-Tyr-Lys-Pro-Asp-Glu-Gly-Lys-Arg-Gly-Asp-Ala-Cys-X_{I}-Gly-Asp-Ser-Gly-Gly-Pro-X₂-Val. Another preferred thrombin peptide derivative for use in the disclosed method comprises the amino acid sequence of SEQ ID NO:19: Asp-Asn-Met-Phe-Cys-Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val-Met-Lys-Ser-Pro-Phe. X₁ is Glu or Gln; X₂ is Phe, Met, Leu, His or Val. The thrombin peptide derivatives of SEQ ID NO:2 and SEQ ID NO:19 can optionally comprise a C-terminal amide and/or acylated N-terminus, as defined above. Preferably, the N-terminus is free (i.e., unsubstituted) and the C-terminus is free (i.e., unsubstituted) or amidated, preferably as a carboxamide (i.e., —C(O)NH₂). Alternatively, N-terminal truncated fragments of these preferred thrombin peptide derivatives, the N-terminal truncated fragments having at least fourteen amino acids, or C-terminal truncated fragments of these preferred thrombin peptide derivatives, the C-terminal truncated fragments having at least eighteen amino acids, can also be used in the disclosed method.

TP508 is an example of a thrombin peptide derivative and is 23 amino acid residues long, wherein the N-terminal amino acid residue Ala is unsubstituted and the COOH of the C-terminal amino acid Val is modified to an amide represented by —C(O)NH₂ (SEQ ID NO:3). Another example of a thrombin peptide derivative comprises the amino acid sequence of SEQ ID NO:6, wherein both N- and C-termini are unsubstituted (“deamide TP508”). Other examples of thrombin peptide derivatives which can be used in the disclosed method include N-terminal truncated fragments of TP508 (or deamide TP508), the N-terminal truncated fragments having at least fourteen amino acids, or C-terminal truncated fragments of TP508 (or deamide TP508), the C-terminal truncated fragments having at least eighteen amino acids.

As used herein, a “conservative amino acid substitution” or a “conservative substitution” in an NPAR agonist is the replacement of an amino acid with another amino acid that has the same net electronic charge and approximately the same size and shape. Amino acids with aliphatic or substituted aliphatic amino acid side chains have approximately the same size when the total number of carbon and heteroatoms in their side chains differs by no more than about four. They have approximately the same shape when the number of branches in their side chains differs by no more than one. Amino acids with phenyl or substituted phenyl groups in their side chains are considered to have about the same size and shape. Listed below are five groups of amino acids. Replacing an amino acid in an NPAR agonist with another amino acid from the same group results in a conservative substitution:
Group I: glycine, alanine, valine, leucine, isoleucine, serine, threonine, cysteine, and non-naturally occurring amino acids with C1-C4 aliphatic or C1-C4 hydroxyl substituted aliphatic side chains (straight chained or monobranched).
Group II: glutamic acid, aspartic acid and non-naturally occurring amino acids with carboxylic acid substituted C1-C4 aliphatic side chains (unbranched or one branch point).
Group III: lysine, ornithine, arginine and non-naturally occurring amino acids with amine or guanidino substituted C1-C4 aliphatic side chains (unbranched or one branch point).
Group IV: glutamine, asparagine and non-naturally occurring amino acids with amide substituted C1-C4 aliphatic side chains (unbranched or one branch point).
Group V: phenylalanine, phenylglycine, tyrosine and tryptophan.

As used herein, a “highly conservative substitution” in a polypeptide is the replacement of an amino acid with another amino acid that has the same functional group in the side chain and nearly the same size and shape. Amino acids with aliphatic or substituted aliphatic amino acid side chains have nearly the same size when the total number of carbon and heteroatoms in their side chains differs by no more than two. They have nearly the same shape when they have the same number of branches in the their side chains. Examples of highly conservative substitutions include valine for leucine, threonine for serine, aspartic acid for glutamic acid and phenylglycine for phenylalanine. Examples of substitutions which are not highly conservative include alanine for valine, alanine for serine and aspartic acid for serine.

Thrombin peptide derivatives retain their monomeric form essentially free of dimers in the presence of a dimerization inhibitor such as a chelating agent or a thiol-containing compound, e.g., greater than 90% free by weight over a two-month time period and preferably greater than 95% free by weight over a two-month time period. The chelating agent and the thiol-containing compound can be used together or separately to prevent or reduce dimerization of thrombin peptide derivatives. An antioxidant optionally can be used in combination with the chelating agent and/or the thiol-containing compound. See Publication No. US2005/0203017 A1, which is hereby incorporated by reference in its entirety.

### Modified Thrombin Peptide Derivatives

In one embodiment of the invention, the NPAR agonists are modified relative to the thrombin peptide derivatives described above, wherein cysteine residues of aforementioned thrombin peptide derivatives are replaced with amino acids having similar size and charge properties to minimize dimerization of the peptides. Examples of suitable amino acids include alanine, glycine, serine, or an S-protected cysteine. Preferably, cysteine is replaced with alanine. The modified thrombin peptide derivatives have about the same biological activity as the unmodified thrombin peptide derivatives. See Publication No. US 2005/0158301 A1, which is hereby incorporated by reference.

It will be understood that the modified thrombin peptide derivatives disclosed herein can optionally comprise C-terminal amides and/or N-terminal acyl groups, as described above. Preferably, the N-terminus of a thrombin peptide derivative is free (i.e., unsubstituted) and the C-terminus is free (i.e., unsubstituted) or amidated, preferably as a carboxamide (i.e., —C(O)NH₂).

In a specific embodiment, the modified thrombin peptide derivative comprises a polypeptide having the amino acid sequence of SEQ ID NO: 4: Arg-Gly-Asp-Ala-Xaa-X₁-Gly-Asp-Ser-Gly-Gly-Pro-X₂-Val, or a C-terminal truncated fragment thereof having at least six amino acids. More specifically, the thrombin peptide derivative comprises the amino acid sequence of SEQ ID NO: 20: Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val or a fragment thereof comprising amino acids 10-18 of SEQ ID NO: 20. Even more specifically, the thrombin peptide derivative comprises the amino acid sequence SEQ ID NO: 5: Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val, or a fragment thereof comprising amino acids 10-18 of SEQ ID NO: 5. Xaa is alanine, glycine, serine or an 5-protected cysteine. X_{I} is Glu or Gln and X₂ is Phe, Met, Leu, His or Val. Preferably X₁ is Glu, X₂ is Phe, and Xaa is alanine. One example of a thrombin peptide derivative of this type is a polypeptide having the amino acid sequence Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val (SEQ ID NO:21). A further example of a thrombin peptide derivative of this type is the polypeptide Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-NH₂ (SEQ ID NO:22). Zero, one, two or three amino acids in the thrombin peptide derivative differ from the amino acid at the corresponding position of SEQ ID NO:4, 20, 5, 21 or 22, provided that Xaa is alanine, glycine, serine or an S-protected cysteine. Preferably, the difference is conservative, as defined for conservative substitutions of NPAR agonists.

In another specific embodiment, the thrombin peptide derivative comprises a polypeptide having the amino acid sequence SEQ ID NO:23: Asp-Asn-Met-Phe-Xbb-Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val-Met-Lys-Ser-Pro-Phe, or a fragment thereof comprising amino acids 6-28. More preferably, the thrombin peptide derivative comprises a polypeptide having the amino acid sequence SEQ ID NO:24: Asp-Asn-Met-Phe-Xbb-Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val-Met-Lys-Ser-Pro-Phe, or a fragment thereof comprising amino acids 6-28. Xaa and Xbb are independently alanine, glycine, serine or an S-protected cysteine. X₁ is Glu or Gln and X₂ is Phe, Met, Leu, His or Val. Preferably X_{I} is Glu, X₂ is Phe, and Xaa and Xbb are alanine. One example of a thrombin peptide derivative of this type is a polypeptide comprising the amino acid sequence Asp-Asn-Met-Phe-Ala-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-Met-Lys-Ser-Pro-Phe (SEQ ID NO:25). A further example of a thrombin peptide derivative of this type is the polypeptide Asp-Asn-Met-Phe-Ala-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-Met-Lys-Ser-Pro-Phe-NH₂ (SEQ ID NO:26). Zero, one, two or three amino acids in the thrombin peptide derivative can differ from the amino acid at the corresponding position of SEQ ID NO:23, 24, 25 or 26. Xaa and Xbb are independently alanine, glycine, serine or an S-protected cysteine. Preferably, the difference is conservative, as defined for conservative substitutions of NPAR agonists.

An “S-protected cysteine” is a cysteine residue in which the reactivity of the thiol moiety, —SH, is blocked with a protecting group. Suitable protecting groups are known in the art and are disclosed, for example, in T. W. Greene and P. G. M. Wuts, Protective Groups in Organic Synthesis, 3^{rd} Edition, John Wiley & Sons, (1999), pp. 454-493, the teachings of which are incorporated herein by reference in their entirety. Suitable protecting groups should be non-toxic, stable in pharmaceutical formulations and have minimum additional functionality to maintain the activity of the thrombin peptide derivative. A free thiol can be protected as a thioether, a thioester, or can be oxidized to an unsymmetrical disulfide. Preferably the thiol is protected as a thioether. Suitable thioethers include, but are not limited to, S-alkyl thioethers (e.g., C₁-C₅ alkyl), and S-benzyl thioethers (e.g, cysteine-S-S-t-Bu). Preferably the protective group is an alkyl thioether. More preferably, the S-protected cysteine is an S-methyl cysteine. Alternatively, the protecting group can be: 1) a cysteine or a cysteine-containing peptide (the “protecting peptide”) attached to the cysteine thiol group of the thrombin peptide derivative by a disulfide bond; or 2) an amino acid or peptide (“protecting peptide”) attached by a thioamide bond between the cysteine thiol group of the thrombin peptide derivative and a carboxylic acid in the protecting peptide (e.g., at the C-terminus or side chain of aspartic acid or glutamic acid). The protecting peptide can be physiologically inert (e.g., a polyglycine or polyalanine of no more than about fifty amino acids optionally interrupted by a cysteine), or can have a desirable biological activity.

### Thrombin Peptide Derivative Dimers

In some aspects of the present invention, the NPAR agonists of the methods are thrombin peptide derivative dimers. See Publication No. 2005/0153893, which is hereby incorporated by reference. The dimers essentially do not revert to monomers and still have about the same biological activity as the thrombin peptide derivatives monomer described above. A “thrombin peptide derivative dimer” is a molecule comprising two thrombin peptide derivatives linked by a covalent bond, preferably a disulfide bond between cysteine residues. Thrombin peptide derivative dimers are typically essentially free of the corresponding monomer, e.g., greater than 95% free by weight and preferably greater than 99% free by weight. Preferably the polypeptides are the same and covalently linked through a disulfide bond.

The thrombin peptide derivative dimers of the present invention comprise the thrombin peptide derivatives described above. Specifically, thrombin peptide derivatives have less than about fifty amino acids, preferably less than about thirty-three amino acids. Thrombin peptide derivatives also have sufficient homology to the fragment of human thrombin corresponding to thrombin amino acid residues 508-530: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6) so that the polypeptide activates NPAR. The thrombin peptide derivative dimers described herein are formed from polypeptides typically having at least six amino acids and preferably from about 12 to about 33 amino acid residues, and more preferably from about 12 to about 23 amino acid residues.

In a specific embodiment, each thrombin peptide derivative comprising a dimer comprises a polypeptide having the amino acid sequence SEQ ID NO:1: Arg-Gly-Asp-Ala-Cys-X₁-Gly-Asp-Ser-Gly-Gly-Pro-X₂-Val, or a C-terminal truncated fragment thereof comprising at least six amino acids. More specifically, each thrombin peptide derivative comprises the amino acid sequence of SEQ ID NO:6: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val, or a fragment thereof comprising amino acids 10-18 of SEQ ID NO: 5. Even more specifically, the thrombin peptide derivative comprises the amino acid sequence SEQ ID NO:2: Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val, or a fragment thereof comprising amino acids 10-18 of SEQ ID NO:2. X_{I} is Glu or Gln and X₂ is Phe, Met, Leu, His or Val. Preferably X₁ is Glu, and X₂ is Phe. One example of a thrombin peptide derivative of this type is a polypeptide comprising the amino acid sequence Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). A further example of a thrombin peptide derivative of this type is a polypeptide having the amino acid sequence Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3). Zero, one, two or three amino acids in the thrombin peptide derivative differ from the amino acid at the corresponding position of SEQ ID NO:6, 1, 2, or 3. Preferably, the difference is conservative, as in conservative substitutions in NPAR agonists.

One example of a thrombin peptide derivative dimer of the present invention is represented by Formula (IV):

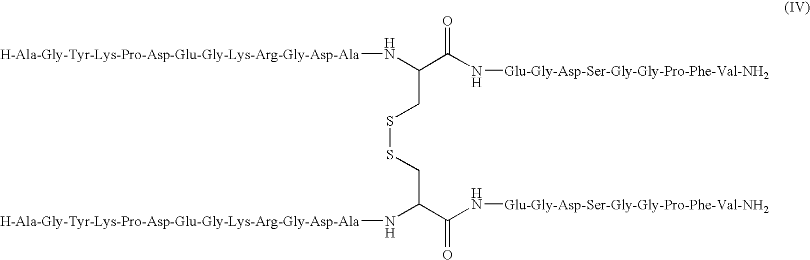

In another specific embodiment, each thrombin peptide derivative comprising a dimer comprises a polypeptide comprising the amino acid sequence SEQ ID NO:27: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr, or a C-terminal truncated fragment thereof having at least twenty-three amino acids. More preferably, each thrombin peptide derivative comprises the amino acid sequence SEQ ID NO:28: Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val-Met-Lys-Ser-Pro-Phe-Asn-Asn-Arg-Trp-Tyr, or a C-terminal truncated fragment thereof comprising at least twenty-three amino acids. X₁ is Glu or Gln and X₂ is Phe, Met, Leu, His or Val. Preferably X₁ is Glu, and X₂ is Phe. One example of a thrombin peptide derivative of this type is a polypeptide comprising the amino acid sequence Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr (SEQ ID NO:27). A further example of a thrombin peptide derivative of this type is a polypeptide comprising the amino acid sequence Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr-NH₂ (SEQ ID NO:29). Zero, one, two or three amino acids in the thrombin peptide derivative differ from the amino acid at the corresponding position of SEQ ID NO:27, 28 or 29. Preferably, the difference is conservative, as defined for conservative substitutions of NPAR agonists.

### NPAR Agonist Antibodies

A particular class of NPAR agonists includes antibodies and antigen-binding fragments that can both bind to and activate the non-proteolytically activated thrombin receptor (NPAR). Agonist antibodies that bind to thrombin receptors have been described in the art. For example, Frost et al. teach that a monoclonal antibody, TR-9, can mimic the effects of thrombin's high affinity interaction with the high affinity thrombin receptor (Frost, G. H., et al., J. Cell Biol. 105 (6 PT. 1):2551-58 (1987)).

Antibodies or antigen-binding fragments thereof that are NPAR agonists can be found by their binding to a complementary peptide that is encoded by the complement of a nucleotide sequence encoding a portion of thrombin. See Molecular Recognition Theory below. The NPAR agonist antibody or antigen-binding fragment binds to a complementary peptide that is encoded by the complement of a nucleotide sequence encoding a portion of thrombin. An NPAR agonist antibody or antigen-binding fragment can be found by its binding to a complementary peptide that is encoded by the complement of a nucleotide sequence encoding a portion of thrombin. In one embodiment, the thrombin or portion thereof (which is encoded by the sense or +RNA strand and is the complement of the RNA strand encoding the complementary peptide to which the antibody or antigen-binding fragment binds) is a mammalian thrombin or a portion of a mammalian thrombin. In another embodiment, the thrombin or portion thereof is a human thrombin or a portion of a human thrombin.

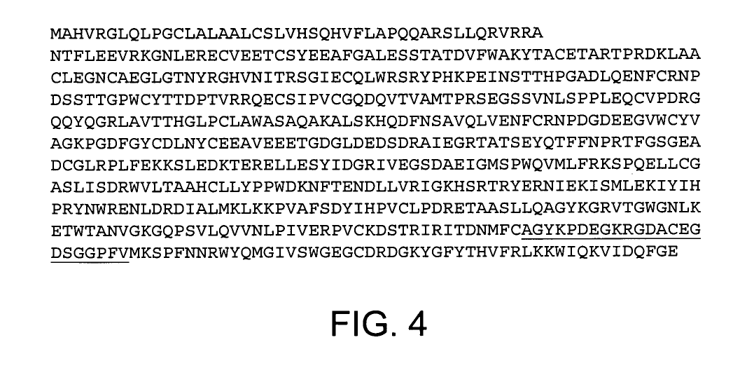

Antibodies or antigen-binding fragments thereof that bind to a complementary peptide, wherein the complementary peptide is encoded by the complement of a nucleotide sequence encoding thrombin or a portion thereof, can be NPAR agonists. In one embodiment, the portion of thrombin (which is encoded by the sense or +RNA strand and is the complement of the RNA strand encoding the complementary peptide to which the antibody or antigen-binding fragment binds) is a thrombin receptor binding domain or a portion thereof. As used herein, a thrombin receptor binding domain or a portion thereof is a segment of thrombin that is capable of selectively binding to the high-affinity non-proteolytically activated thrombin receptor (NPAR). Such thrombin receptor binding domains contain a portion of a domain (represented by amino acid residues 517-520 of human thrombin; see FIG. 4 depicting the amino acid sequence of human prothrombin (SEQ ID NO:12) with a sequence homologous to the tripeptide cell binding domain of fibronectin, Arg-Gly-Asp. In a particular embodiment, the thrombin receptor binding domain or portion thereof comprises the amino acid sequence AGYKPDEGKRGDACEGDSGGPFV (i.e., amino acids 508-530 of human thrombin (SEQ ID NO:6)). In another embodiment, the thrombin receptor binding domain or portion thereof is a portion of the thrombin receptor binding domain and comprises the amino acid sequence EGKRGDACEG (SEQ ID NO:7).

As described herein, complementary peptides of domains of thrombin that are encoded by both the 5′-3′ sequence of the antisense RNA strand and the 3′-5′ sequence of the antisense RNA strand can be used to produce the NPAR agonist antibodies and antigen-binding domains of the invention. Therefore, in one embodiment, the complementary peptide (to which the antibodies and antigen-binding fragments bind) is encoded by the 5′-3′ sequence of the antisense RNA strand. In another embodiment, the complementary peptide is encoded by the 3′-5′ sequence of the antisense RNA strand.

In one example, a complementary peptide (to which the NPAR agonist antibodies and antigen-binding fragments of the invention bind) comprises the amino acid sequence KGSPTVTFTGIPCFPFIRLVTS (AC-23; SEQ ID NO:30). In another example, the complementary peptide comprises the amino acid sequence KGSPTVTFTGIPSFPFIRLVTS (23C53; SEQ ID NO:31). In yet another example, the complementary peptide comprises the amino acid sequence TFTGIPSFPF (C1053; SEQ ID NO:32). In still another example, the complementary peptide comprises the amino acid sequence RPMFGLLPFAPLRTLPLSPPGKQ [AC-23rev (SEQ ID NO:33), which is the complementary 5′-3′ peptide corresponding to AC-23]. In still a further example, the complementary peptide comprises the amino acid sequence LPFAPLRTLP [C1053rev (SEQ ID NO:34), which is the complementary 5′-3′ peptide corresponding to C1053].

One example of an NPAR agonist antibody or an antigen-binding fragment thereof binds to a cysteine-altering complementary peptide comprising the amino acid sequence KGSPTVTFTGIPSFPFIRLVTS (23C53; SEQ ID NO:31). 23C53, which differs from AC-23 by a single amino acid, is the complementary peptide of TP508, except that it possesses a single amino acid alteration from Cys to Ser.

In binding experiments using biotin-conjugated thrombin, thrombin was found to bind specifically to AC23 and 23C53. Half maximal binding of biotin-labeled thrombin to AC-23 was 4.8±0.2 nM (n=2±SD).

Addition of TP508 inhibited specific binding of biotin-labeled thrombin to AC-23. Up to 60% of the binding of thrombin to AC-23 can be inhibited by the addition of TP508. Therefore, both thrombin and TP508 bind to the complementary peptide, AC-23. This suggests that AC-23 has a three-dimensional structure that is similar to the thrombin-TP508 receptor on cells. Antibodies to AC-23 and other complementary peptides of thrombin can therefore be used to characterize the thrombin binding site that is activated by TP508, and can be used in the therapeutic and other methods described herein.

In addition to the thrombin receptor binding domain, the stimulatory (agonistic) thrombin polypeptide derivatives possess a domain (represented by amino acid residues 519-530 of human thrombin) with a high degree of homology to a number of serine esterases. However, the inhibitory (antagonistic) thrombin polypeptide derivatives do not include the serine esterase domain.

Thrombin peptide derivatives from amino acid residues 508-530 of human thrombin have been described for promoting thrombin receptor mediated cell stimulation. In addition, stimulatory (agonistic) thrombin polypeptide derivatives containing both fibronectin- and serine protease-homologous domains (residues 508 to 530 of human thrombin) bind to thrombin receptors with high-affinity and substitute for DIP-alpha-thrombin as an initiator of receptor occupancy-related mitogenic signals. (DIP-alpha-thrombin is a proteolytically inactive derivative of thrombin that retains receptor binding activity.) In contrast, inhibitory (antagonistic) thrombin polypeptide derivatives containing only the fibronectin-homologous domain (p517-520) (but not the serine protease-homologous domain) bind to the thrombin receptor without inducing mitogenesis. An intermediate thrombin peptide derivative (p519-530) retains the ability to mediate mitogenesis but to a much lesser degree than p508-530.

### Molecular Recognition Theory

Blalock and Smith (1984) observed that the hydropathic character of an amino acid residue is related to the identity of the middle letter of the triplet codon from which it is transcribed (Blalock, J. E., and Smith, E. M., Biochem. Biophys. Res. Commun. 12: 203-07 (1984)). Specifically, a triplet codon with thymine (T) as its middle base codes for a hydrophobic residue while adenine (A) codes for a hydrophilic residue. A triplet codon with middle bases cytosine (C) or guanine (G) encode residues that are relatively neutral and with similar hydropathy scores. Hydropathy is an index of the affinity of an amino acid for a polar environment; hydrophilic residues yielding a more negative score, while hydrophobic residues exhibit more positive scores. Kyte and Doolittle (1982) conceived a hydropathy scale that is widely used (Kyte, J., and Doolittle, R. F., J. Mol. Biol. 5:105-32 (1982)). The observed relationship between the middle base of a triplet codon and residue hydropathy entails that peptides encoded by complementary DNA will exhibit complementary, or inverted, hydropathic profiles. It was proposed that because two peptide sequences encoded in complementary DNA strands display inverted hydropathic profiles, they may form amphipathic secondary structures, and bind to one another (Bost, K. L., et al., Proc. Natl. Acad. Sci. USA 82:1372-75 (1985)). Complementary peptides have been reported to form binding complexes with their “sense” peptide counterparts for a number of different systems (Root-Bernstein, R. S., and Holsworthy, D. D., J. Theor. Biol. 190:107-19 (1988)). For example, Gho and Chae describe peptide antagonists of human angiogenin that are complementary peptides encoded by the antisense RNA sequence corresponding to the receptor binding site of angiogenin (Gho, Y. S, and Chae, C. B. J. Biol. Chem. 272(39):24294-99 (1997)). Ghiso et al. describe a peptide complementary to a region of cystatin C that exhibits inhibitory activity (Ghiso, J., et al., Proc. Natl. Acad. Sci. USA 87(4):1288-91 (1990)), and Bost and Blalock describe the production of anti-idiotypic antibodies by immunization with a pair of complementary peptides (Bost. K. L., and Blalock, J. E., J. Molec. Recognit. 1:179-83 (1989)).

The scope of this analysis for explaining the interactions between proteins was further developed by Blalock to propose a Molecular Recognition Theory (MRT) (Bost, K. L., et al., Proc. Natl. Acad. Sci. USA 82:1372-75 (1985); Blalock, J. E., Nature Med. 1:876-78 (1995)). This theory suggests that a “molecular recognition” code of interaction exists between peptides that are encoded by complementary strands of DNA, based on the observation that such peptides will exhibit inverted hydropathic profiles. MRT has proved successful for predicting particular binding interactors.

Blalock suggested that it is the linear pattern of amino acid hydropathy scores in a sequence (rather than the combination of specific residue identities), that defines the secondary structure environment. Furthermore, he suggested that sequences with inverted hydropathic profiles are complementary in shape by virtue of inverse forces that determine their steric relationships.

### Deriving a Complementary Peptide in the 3′-5′ Reading Frame

As a corollary to his original work, Blalock contended that as well as reading a complementary codon in the usual 5′-3′ direction, reading a complementary codon in the 3′-5′ direction would also yield amino acid sequences that displayed opposite hydropathic profiles (Bost, K. L., et al., Proc. Natl. Acad. Sci. USA 82:1372-75 (1985)). This follows from the observation that the middle base of a triplet codon determines the hydropathy index of the residue it codes for, and therefore reading a codon in the reverse direction may change the identity, but not the hydropathic nature of the coded amino acid (Table 4).

TABLE 4
The relationships between amino acids and the residues encoded in the complementary strand
The relationships between amino acids and the residues encoded in the complementary strand reading 3′-5′

| | | Comple- | Comple- | | | Comple- | Comple- |
|---|---|---|---|---|---|---|---|
| Amino | | mentary | mentary | Amino | | mentary | mentary |
| Acid | Codon | codon | Amino acid | Acid | codon | codon | Amino acid |
| Alanine | GCA | CGU | Arginine | Serine | UCA | AGU | Serine |
| | GCG | CGC | | | UCC | AGQ | Arginine |
| | GCC | CGG | | | UCG | AGC | Serine |
| | GCU | CGA | | | UCU | AGA | Arginine |
| Arginine | CGG | GCC | Alanine | | AGC | UCG | Serine |
| | CGA | GCU | Alanine | | AGU | UCA | Serine |
| | CGC | GCG | Alanine | Glutamine | CAA | GUU | Valine |
| | CGU | GCA | Alanine | | CAG | GUC | Valine |
| | AGG | UCC | Serine | Glycine | GGA | CCU | Proline |
| | AGA | UCU | Serine | | GGC | CCG | Proline |
| Aspartic | GAC | GUC | Valine | | GGU | CCA | Proline |
| Acid | GAU | AUC | Isoleucine | | GGG | CCC | Proline |
| Asparagine | AAC | UUG | Leucine | Histidine | CAC | GUG | Valine |
| | AAU | UUA | Leucine | | CAU | GUA | Valine |
| Cysteine | UGU | ACA | Threonine | Isoleucine | AUA | UAU | Tyrosine |
| | UGC | ACG | Threonine | | AUC | UAG | Stop |
| Glutamic | GAA | CUU | Leucine | | AUU | UAA | Stop |
| Acid | GAG | CUG | Leucine | Leucine | CUG | GAC | Asp |
| Lysine | AAA | UUU | Phenylalanine | | CUC | GAG | Glutamic acid |
| | AAG | UUC | Phenylalanine | | CUU | GAA | Glutamic Acid |
| Methionine | AUG | UAC | Tyrosine | | UUA | AAU | Asparagine |
| Phenylalanine | UUU | AAA | Lysine | | CUA | GAU | Aspartic Acid |
| | UUC | AAG | Lysine | | UUG | AAC | Asparagine |
| Proline | CCA | GGU | Glycine | | CUG | GAC | Aspartic Acid |
| | CCC | GGG | Glycine | Threonine | ACA | UGU | Cysteine |
| | CCU | GGA | Glycine | | ACG | UGC | Cysteine |
| | CCG | GGC | Glycine | | ACC | UGG | Tryptophan |
| | | | | | ACU | UGA | Stop |
| | | | | Tryptophan | UGG | ACC | Threonine |
| | | | | Tyrosine | UAC | AUG | Methionine |
| | | | | | UAU | AUA | Isoleucine |
| | | | | Valine | GUA | CAU | Histidine |
| | | | | | GUG | CAC | Histidine |
| | | | | | GUC | CAG | Glutamine |
| | | | | | GUU | CAA | Glutamine |

### Antibodies and Antibody Producing Cells

NPAR agonists as referred to herein encompass antibodies and antigen-binding fragments thereof that bind to the complementary peptides described herein and activate the non-proteolytically activated thrombin receptor. The antibodies as referred to herein can be polyclonal or monoclonal, and the term “antibody” is intended to encompass both polyclonal and monoclonal antibodies. The terms polyclonal and monoclonal refer to the degree of homogeneity of an antibody preparation, and are not intended to be limited to particular methods of production. In one embodiment, the antibody or antigen-binding fragment is a monoclonal antibody or antigen-binding fragment thereof. The term “monoclonal antibody” or “monoclonal antibody composition” as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

The term “antibody” as used herein also encompasses functional fragments of antibodies, including fragments of chimeric, humanized, primatized, veneered or single chain antibodies. Functional fragments include antigen-binding fragments of antibodies that bind to the complementary peptides, wherein complementary peptides are encoded by the complement of a nucleotide sequence encoding thrombin or a portion thereof. For example, antibody fragments capable of binding to a complementary peptide, include, but are not limited to Fv, Fab, Fab′ and F(ab′)₂ fragments. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH_{I} domain and hinge region of the heavy chain.

Single chain antibodies, and chimeric, humanized or primatized (CDR-grafted), or veneered antibodies, as well as chimeric, CDR-grafted or veneered single chain antibodies, comprising portions derived from different species, are also encompassed by the term antibody. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity can be selected (e.g., from a phage library; see e.g., Krebber et al., U.S. Pat. No. 5,514,548; Hoogenboom et al., WO 93/06213).

The antibody can be a humanized antibody comprising one or more immunoglobulin chains [e.g., an antibody comprising a complementarity-determining region (CDR) of nonhuman origin (e.g., one or more CDRs derived from an antibody of nonhuman origin)] and a framework region derived from a light and/or heavy chain of human origin (e.g., CDR-grafted antibodies with or without framework changes)]. In one embodiment, the antibody or antigen-binding fragment thereof comprises the light chain CDRs (CDR1, CDR2 and CDR3) and heavy chain CDRs (CDR1, CDR2 and CDR3) of a particular immunoglobulin. In another embodiment, the antibody or antigen-binding fragment further comprises a human framework region.

Antibodies that are specific for a complementary peptide, wherein the complementary peptide is encoded by the complement of a nucleotide sequence encoding thrombin or a portion thereof, can be raised against an appropriate immunogen, such as a synthetic or recombinant complementary peptide or a portion thereof. Antibodies can also be raised by immunizing a suitable host (e.g., mouse) with transfected cells that express a complementary peptide. Such cells can also be used in a screen for an antibody that binds thereto (See e.g., Chuntharapai et al., J. Immunol., 152: 1783-1789 (1994); Chuntharapai et al., U.S. Pat. No. 5,440,021).

Preparation of Immunizing Antigen, and Polyclonal and Monoclonal Antibody production can be performed using any suitable technique (e.g., as exemplified herein). A variety of methods have been described (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991)). Generally, a hybridoma is produced by fusing a suitable immortal cell line (e.g., a myeloma cell line, such as SP2/0, P3X63Ag8.653 or a heteromyeloma) with antibody-producing cells. Antibody-producing cells can be obtained from the peripheral blood or, preferably the spleen or lymph nodes, of humans or other suitable animals immunized with a complementary peptide. The fused cells (hybridomas) can be isolated using selective culture conditions, and cloned by limiting dilution. Cells that produce antibodies with the desired specificity can be selected by a suitable assay (e.g., ELISA).

Other suitable methods of producing or isolating antibodies of the requisite specificity (e.g., human antibodies or antigen-binding fragments) can be used, including, for example, methods that select recombinant antibody from a library (e.g., a phage display library). Transgenic animals capable of producing a repertoire of human antibodies (e.g., Xenomouse® (Abgenix, Fremont, Calif.)) can be produced using suitable methods (see e.g., Jakobovits et al., Proc. Natl. Acad. Sci. USA, 90: 2551-2555 (1993); Jakobovits et al., Nature, 362: 255-258 (1993)). Additional methods that are suitable for production of transgenic animals capable of producing a repertoire of human antibodies have been described (e.g., Lonberg et al., U.S. Pat. No. 5,545,806; Surani et al., U.S. Pat. No. 5,545,807; Lonberg et al., WO 97/13852).

Bispecific antibodies, or functional fragments thereof (e.g., F(ab′)₂) can bind to a complementary peptide as described herein and at least one other antigen (e.g., a tumor antigen, a viral antigen). Bispecific antibodies can be secreted by triomas and hybrid hybridomas. Generally, triomas are formed by fusion of a hybridoma and a lymphocyte (e.g., antibody-secreting B cell) and hybrid hybridomas are formed by fusion of two hybridomas. Each of the fused cells (i.e., hybridomas, lymphocytes) produces a monospecific antibody. However, triomas and hybrid hybridomas can produce an antibody containing antigen-binding sites that recognize different antigens. The supernatants of triomas and hybrid hybridomas can be assayed for bispecific antibody using a suitable assay (e.g., ELISA), and bispecific antibodies can be purified using conventional methods. (see, e.g., U.S. Pat. No. 5,959,084 (Ring et al.), U.S. Pat. No. 5,141,736 (Iwasa et al.), U.S. Pat. Nos. 4,444,878, 5,292,668, 5,523,210 (all to Paulus et al.) and U.S. Pat. No. 5,496,549 (Yamazaki et al.)).

### Methods of Promoting Cardiac Tissue Revascularization and Methods of Promoting Vascular Endothelial Cell Migration and Proliferation

Administration of a combination comprising therapeutic agents (e.g., NPAR agonist and angiogenic growth factor) includes simultaneous (concurrent) administration as well as consecutive administration in any order. The agents can be administered together in one composition or can be administered in separate compositions over a period of time of treatment. Separate compositions can be administered by the same or by different routes of administration, on the same or on different schedules.

An NPAR agonist and angiogenic growth factor can be administered to a subject in combination as the only two biologically active agents, or one or more NPAR agonists and one or more angiogenic growth factors can be administered with one or more additional therapeutic agents or procedures. The administration of a combination of agents includes 1) coadministration, using separate formulations or a single pharmaceutical formulation, and 2) consecutive administration in any order. For example, the NPAR agonist may precede, follow, or alternate with administration of the angiogenic growth factor, or may be given simultaneously therewith. Use of multiple agents is also included in the invention. In one embodiment, there is a time period while both (or all) active agents simultaneously exert their biological activities. In a combination therapy regimen, the NPAR agonist and angiogenic growth factor are administered such that the combination is in a therapeutically effective amount.

A “therapeutically effective amount” of a combination is the quantity of NPAR agonist and the quantity of angiogenic growth factor which results in greater cardiac tissue repair, greater cardiac revascularization, greater endothelial cell migration or greater endothelial cell proliferation than is observed in the absence of administration of the combination, as can be evaluated by methods available in the art. The combination of agents is administered for a sufficient period of time to achieve the desired therapeutic effect. The amounts administered will depend on the health, size, weight, age and sex of the subject, the nature of the vascular defect or condition to be treated. Typically, between about 0.1 μg per day and about 1 mg per day of NPAR agonist (preferably between about 1 μg per day and about 100 μg per day) is administered in the combination. The angiogenic growth factor can be administered in a dosage which can be determined by one of ordinary skill in the art, according to the nature of the vascular defect or condition, the site of treatment, the age, sex, weight and other conditions of the subject. Appropriate dosages of angiogenic growth factor in the combination are 1 mg/kg to 50 mg/kg (e.g., 0.1-20 mg/kg). In certain instances, it may be advantageous to co-administer one or more pharmacologically active agents in addition to an angiogenic growth factor and NPAR agonist.

The compositions used in the present invention to stimulate cardiac revascularization, stimulate vascular endothelial cell migration or stimulate vascular endothelial cell proliferation can additionally comprise a pharmaceutical carrier suitable for local administration in which the NPAR agonist and/or angiogenic factor is dissolved or suspended. Standard pharmaceutical formulation techniques can be employed, such as those described in Remington's Pharmaceutical Sciences, Mack Publishing Company, Easton, Pa. Examples of pharmaceutically acceptable carriers include, for example, saline, aerosols, commercially available inert gels, or liquids supplemented with albumin, methyl cellulose or a collagen matrix.

In some embodiments, the NPAR agonist and/or angiogenic factor are administered in a sustained release formulation. Polymers are often used to form sustained release formulations. Examples of these polymers include poly α-hydroxy esters such as polylactic acid/polyglycolic acid homopolymers and copolymers, polyphosphazenes (PPHOS), polyanhydrides and poly(propylene fumarates).

Polylactic acid/polyglycolic acid (PLGA) homo and copolymers are well known in the art as sustained release vehicles. The rate of release can be adjusted by the skilled artisan by variation of polylactic acid to polyglycolic acid ratio and the molecular weight of the polymer (see Anderson, et al., Adv. Drug Deliv. Rev. 28:5 (1997), the entire teachings of which are incorporated herein by reference). The incorporation of poly(ethylene glycol) into the polymer as a blend to form microparticle carriers allows further attenuation of the release profile of the active ingredient (see Cleek et al., J. Control Release 48:259 (1997), the entire teachings of which are incorporated herein by reference). PGLA microparticles are often mixed with pluronic gels or collagen to prevent aggregation and to make the microparticles suitable for direct injection.

PPHOS polymers contain alternating nitrogen and phosphorous with no carbon in the polymer backbone, as shown below in Structural Formula (I):

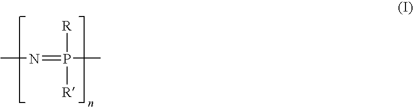

The properties of the polymer can be adjusted by suitable variation of side groups R and R′ that are bonded to the polymer backbone. For example, the degradation of and drug release by PPHOS can be controlled by varying the amount of hydrolytically unstable side groups. With greater incorporation of either imidazolyl or ethylglycinato substituted PPHOS, for example, an increase in degradation rate is observed (see Laurencin et al., J Biomed Mater. Res. 27:963 (1993), the entire teachings of which are incorporated herein by reference), thereby increasing the rate of drug release.

Polyanhydrides, shown in Structural Formula (II), have well defined degradation and release characteristics that can be controlled by including varying amounts of hydrophobic or hydrophilic monomers such as sebacic acid and 1,3-bis(p-carboxyphenoxy)propane (see Leong et al., J. Biomed. Mater. Res. 19:941 (1985), the entire teachings of which are incorporated herein by reference).

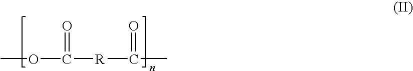

Poly(propylene fumarates) (PPF) are highly desirable biocompatible implantable carriers because they are an injectable, in situ polymerizable, biodegradable material. “Injectable” means that the material can be injected by syringe through a standard needle used for injecting pastes and gels. PPF, combined with a vinyl monomer (N-vinyl pyrrolidinone) and an initiator (benzoyl peroxide), forms an injectable solution that can be polymerized in situ (see Suggs et al., Macromolecules 30:4318 (1997), Peter et al., J. Biomater. Sci. Poly., Ed. 10:363 (1999) and Yaszemski et al., Tissue Eng. 1:41 (1995), the entire teachings of which are incorporated herein by reference).

The combinations of NPAR agonist and angiogenic growth factor described herein can be employed to induce angiogenic proliferation and migration of endothelial cells resulting in formation of new capillaries and collateral vessels to help restore function to damaged or ischemic heart tissue. These combinations can preferably be directly injected into or applied to heart tissue during open chest procedures, for example, for bypass surgery or for insertion of ventricular assist devices, or the combination can be delivered by catheter injection into the heart in one or more compositions. The combination can be in one or more water soluble compositions or can be administered in one or more sustained release formulations.

Endothelial cell proliferation, such as that which occurs in angiogenesis, is also useful in preventing or inhibiting restenosis following balloon angioplasty. The balloon angioplasty procedure often injures the endothelial cells lining the inner walls of blood vessels and disrupts the integrity of the vessel wall. Smooth muscle cells and inflammatory cells often infiltrate into the injured blood vessels causing a secondary obstruction in a process known as restenosis. Stimulation of the proliferation and migration of the endothelial cells located at the periphery of the balloon-induced damaged area in order to cover the luminal surface of the vessel with a new monolayer of endothelial cells would potentially restore the original structure of the blood vessel.

Endothelialization comprises re-endothelialization after angioplasty, to reduce, inhibit or prevent restenosis. Those of skill in the art will recognize that patients treated according to the methods of the present invention may be treated with or without a stent.

Balloon angioplasty is a common treatment of ischemic heart disease which involves the inflation of a balloon in a clogged blood vessel in order to open the blocked blood vessel. Unfortunately, this method of treatment results in injury to the endothelial cells lining the inner walls of blood vessels often leading to restenosis. The peptides described herein can be employed to induce proliferation and migration of the endothelial cells located at the periphery of the balloon induced damaged area in order to cover the luminal surface of the vessel with a new monolayer of endothelial cells, hoping to restore the original structure of the blood vessel. Coronary angioplasty is frequently accompanied by deployment of an intravascular stent to help maintain vessel function and avoid restenosis. Stents have been coated with heparin to prevent thrombosis until the new channel formed by the stent can endothelialize. The combinations of NPAR agonist and angiogenic growth factor described herein can be applied directly to the stent, using methods known to those of skill in the art. The methods of the invention include locally applying to an occluded or damaged blood vessel or systemically administering to a subject undergoing or who has undergone balloon angioplasty, with or without stent placement, any of the combinations of NPAR agonist and angiogenic growth factor described herein. The treatment can enhance endothelialization of the vessel or vessel wall and/or modulate other processes to inhibit or reduce thrombosis and restenosis.

Part of the invention is a method of inhibiting restenosis following balloon angioplasty in a subject, the method comprising administering to the subject a combination in a therapeutically effective amount, the combination comprising one or more angiogenic growth factors, and one or more agonists of the non-proteolytically activated thrombin receptor, wherein the angiogenic growth factor is selected from the group consisting of: human VEGF-A, human VEGF-B, human VEGF-C, human VEGF-D, VEGF-E [Orf virus (D1701)], VEGF-E [Orf virus (NZ2)], VEGF-E_{N27}PlGF, VEGF-E/PlGF, human placental growth factor (PlGF), human platelet derived growth factor D (PDGFD), human platelet derived growth factor alpha (PDGF-α), human platelet derived growth factor 2 (PDGF2), human platelet derived growth factor C (PDGFC), angiogenin, angiopoietin-1, Del-1, acidic fibroblast growth factor (aFGF), basic fibroblast growth factor (bFGF), fibroblast growth factor 4 (FGF 4), follistatin, granulocyte colony-stimulating factor (G-CSF), hepatocyte growth factor (HGF), scatter factor (SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), thymosin beta 4 (Tβ4), connective tissue growth factor, osteopontin, and insulin growth factor (IGF-1).

Another part of the invention is a method of inhibiting vascular occlusion in a patient, the method comprising administering to the subject a combination in a therapeutically effective amount, the combination comprising one or more angiogenic growth factors, and one or more agonists of the non-proteolytically activated thrombin receptor, wherein the angiogenic growth factor is selected from the group consisting of: human VEGF-A, human VEGF-B, human VEGF-C, human VEGF-D, VEGF-E [Orf virus (D1701)], VEGF-E [Orf virus (NZ2)], VEGF-E_{N27}PlGF, VEGF-E/PlGF, human placental growth factor (PlGF), human platelet derived growth factor D (PDGFD), human platelet derived growth factor alpha (PDGF-α), human platelet derived growth factor 2 (PDGF2), human platelet derived growth factor C (PDGFC), angiogenin, angiopoietin-1, Del-1, acidic fibroblast growth factor (aFGF), basic fibroblast growth factor (bFGF), fibroblast growth factor 4 (FGF 4), follistatin, granulocyte colony-stimulating factor (G-CSF), hepatocyte growth factor (HGF), scatter factor (SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), thymosin beta 4 (Tβ4), connective tissue growth factor, osteopontin, and insulin growth factor (IGF-1).

A “subject” is preferably a human (e.g., a “patient”), but can also be an animal in need of treatment, e.g., companion animals (e.g., dogs, cats, and the like), farm animals (e.g., cows, pigs, horses and the like) and laboratory animals (e.g., rats, mice, rabbits, guinea pigs and the like).

Thrombin peptide derivatives and modified thrombin peptide derivatives can be synthesized by solid phase peptide synthesis (e.g., BOC or FMOC) method, by solution phase synthesis, or by other suitable techniques including combinations of the foregoing methods. The BOC and FMOC methods, which are established and widely used, are described in Merrifield, J. Am. Chem. Soc. 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37: 3404 (1972); and Gauspohl, H. et al., Synthesis, 5: 315 (1992)). The teachings of these six articles are incorporated herein by reference in their entirety.

Thrombin peptide derivative dimers can be prepared by oxidation of the monomer. Thrombin peptide derivative dimers can be prepared by reacting the thrombin peptide derivative with an excess of oxidizing agent. A well-known suitable oxidizing agent is iodine.

A “non-aromatic heterocyclic group” as used herein, is a non-aromatic carbocyclic ring system that has 3 to 10 atoms and includes at least one heteroatom, such as nitrogen, oxygen, or sulfur. Examples of non-aromatic heterocyclic groups include piperazinyl, piperidinyl, pyrrolidinyl, morpholinyl, thiomorpholinyl.

The term “aryl group” includes both carbocyclic and heterocyclic aromatic ring systems. Examples of aryl groups include phenyl, indolyl, furanyl and imidazolyl.

An “aliphatic group” is a straight chain, branched or cyclic non-aromatic hydrocarbon. An aliphatic group can be completely saturated or contain one or more units of unsaturation (e.g., double and/or triple bonds), but is preferably saturated, i.e., an alkyl group. Typically, a straight chained or branched aliphatic group has from 1 to about 10 carbon atoms, preferably from 1 to about 4, and a cyclic aliphatic group has from 3 to about 10 carbon atoms, preferably from 3 to about 8. Aliphatic groups include, for example, methyl, ethyl, n-propyl, iso-propyl, n-butyl, sec-butyl, tert-butyl, pentyl, cyclopentyl, hexyl, cyclohexyl, octyl and cyclooctyl.

Suitable substituents for an aliphatic group, an aryl group or a non-aromatic heterocyclic group are those which do not significantly lower therapeutic activity of the NPAR agonist, for example, those found on naturally occurring amino acids. Examples include —OH, a halogen (—Br, —Cl, —I and —F), —O(Rₑ), —O—CO—(Rₑ), —CN, —NO₂, —COOH, ═O, —NH₂—NH(Rₑ), —N(Rₑ)₂, —COO(Rₑ), —CONH₂, —CONH(Rₑ), —CON(Rₑ)₂, —SH, —S(Rₑ), an aliphatic group, an aryl group and a non-aromatic heterocyclic group. Each Rₑ is independently an alkyl group or an aryl group. A substituted aliphatic group can have more than one substituent.

As used in this specification and the appended claims, the singular forms a, an and the include plural referents unless the content clearly dictates otherwise.

### EXAMPLES

### Example 1

### TP508 Potentiates the Ability of VEGV to Signal eNOS Phosphorylation

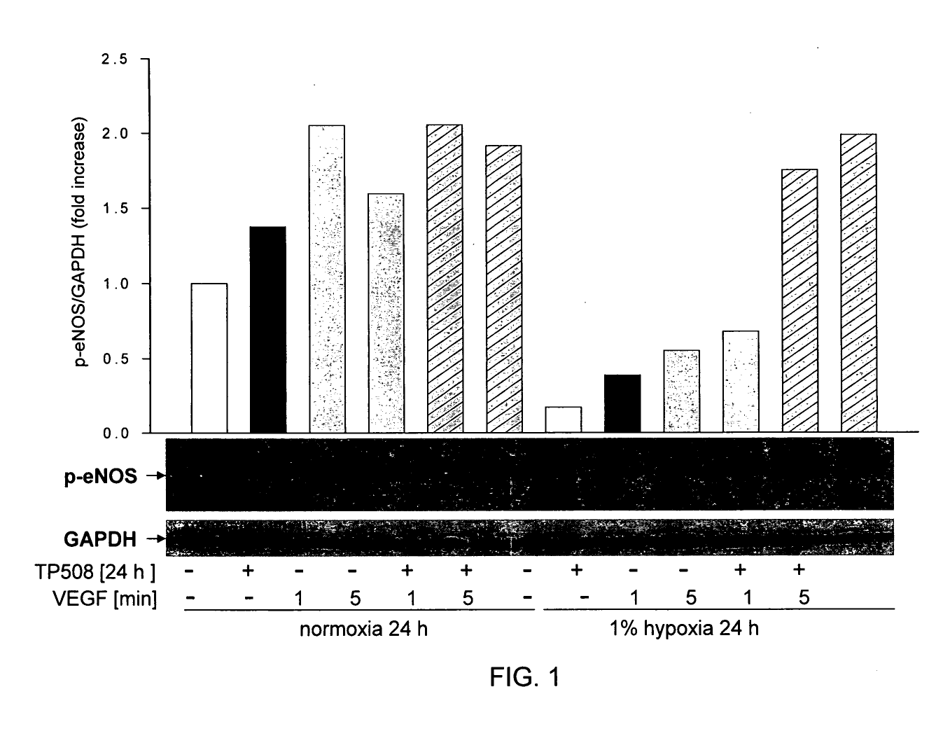

Human coronary artery endothelial (HCAE) cells (Lonza Walkersville, Inc., Walkersville, Md.) were cultured in the presence or absence of TP508 [50 μg/ml] in normoxic and hypoxic [1% O₂] conditions for 24 h and then stimulated with the angiogenic growth factor, human VEGF [50 ng/ml] for 1 or 5 min. Human VEGF-induced eNOS activation was determined by Western blotting using an antibody recognizing the activated form of eNOS (phosphorylated at S1177) (Cell Signaling, Danvers, Mass.). The membrane was re-probed with anti-GAPDH (glyceraldehyde-3-phosphate dehydrogenase) antibody to show equal protein loading. A bar graph representing densitometric analysis of the activated eNOS Western blot after different treatments is shown in FIG. 1.

As shown in FIG. 1, in normoxic cells, human VEGF induces transient phosphorylation of eNOS on serine 1177 to activate the enzyme which is maximum at 1 minute (2-fold) and has declined after 5 minutes stimulation. If cells were pretreated with TP508 prior to human VEGF stimulation, the phosphorylation of eNOS was prolonged and remained near maximum stimulation for 5 minutes. Thus, TP508 potentiates the ability of human VEGF to signal eNOS phosphorylation by extending the period of maximal stimulation.

In hypoxic cells (cultured in 1% O₂ for 24 hours), the level of human VEGF-stimulated eNOS phosphorylation is decreased ˜4 fold at 1 min treatment compared to normoxic cells. Thus, hypoxia significantly reduces human VEGF-stimulated activation of eNOS. However, hypoxic cells pretreated with TP508 showed human VEGF-induced activation of eNOS at levels equivalent to that seen in normoxic cells. Thus, TP508 treatment of hypoxic cells restores the ability of human VEGF to stimulate eNOS activation to the level observed in normoxic cells.

### Example 2

### TP508 Enhances Endothelial Cell Migration Towards VEGF

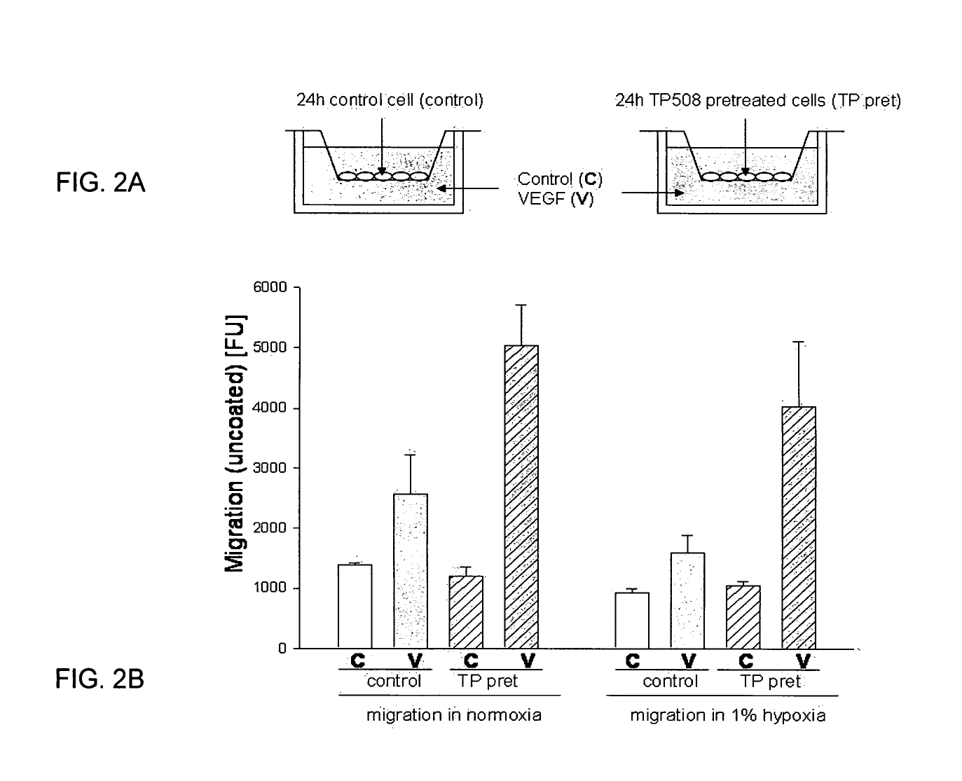

The ability of a test substance to attract endothelial cells and stimulate their migration through pores in the membrane is one of several tests to determine the angiogenic potential of test substances. FIG. 2A shows the design of experiments to measure migration of endothelial cells toward a chemoattractant. Prior to migration assay, cells were cultured with or without TP508 to determine the effect of TP508 on endothelial migration.

Human coronary artery endothelial (HCAE) cells (Lonza Walkersville, Inc., Walkersville, Md.) were cultured in the absence (control) or presence of TP508 [50 μg/ml] (“TP pret” in FIG. 2A and FIG. 2B) for 24 hours. Transmembrane cell migration assays were performed using BD FluoroBlok inserts (BD Bioscience, Bedford, Mass.) as described by the vendor. Control or TP508 pretreated cells were added into the top of the inserts. Human VEGF [10 ng/ml] (V) or medium alone (C) was added to the lower chamber of the insert plate as a chemoattractant. Endothelial migration was performed in normoxic or 1% hypoxic conditions. After a 22-hour incubation, cells were labeled post-migration with Calcein AM and measured by detecting the fluorescence of the cells that migrated to the underside of the insert membrane.

FIG. 2B shows the effect of TP508 treatment on migration of endothelial cells toward the angiogenic factor human VEGF (human recombinant VEGF-A 165, R&D System, Minneapolis, Minn.).

The results show that human VEGF stimulates normal control endothelial cell migration by ˜2 fold relative to media control cells when assayed in normoxic conditions (180%) and slightly less (˜150%) under hypoxic conditions relative to media control cells. Endothelial cells that were pretreated with TP508 showed cell migration toward human VEGF ˜5-fold and ˜4 fold relative to media controls when cells were assayed under normoxic and hypoxic conditions, respectively. TP508 pretreatment, thus, enhances endothelial migration toward human VEGF 2- to 3-fold relative to untreated control cells. Since this cell migration assay is one measure of the angiogenic potential of cells, these results demonstrate that TP508 treatment more than doubles the angiogenic potential of human VEGF for endothelial cells under normoxic conditions as well as under hypoxic conditions where angiogenic responses to human VEGF are diminished.

### Example 3

### TP508 Increases Angiogenic Response of Endothelial Cells Toward Human VEGF

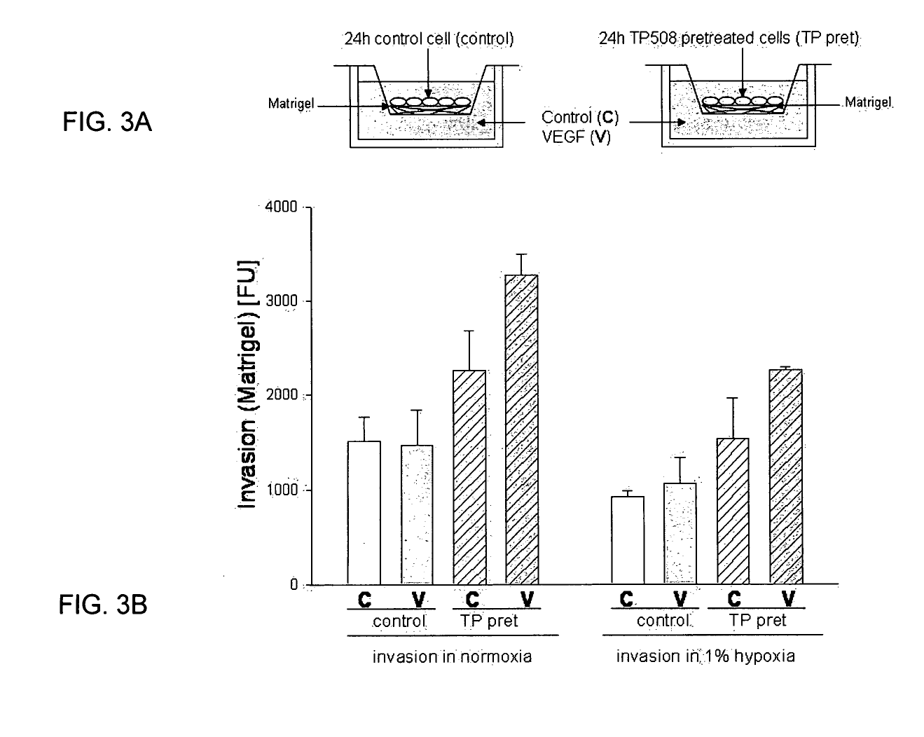

Invasion of endothelial cells through a Matrigel matrix is one of many assays used to determine the angiogenic potential of test substances and is thought to be more predictive of angiogenesis in vivo than a simple chemotactic assay through open membrane pores since the cells must degrade and invade the matrix to move into and through the pores in the membrane. FIG. 3A shows the design of experiments to measure invasion of endothelial cells through Matrigel toward a chemoattractant.

Human coronary artery endothelial (HCAE) cells (Lonza Walkersville, Inc., Walkersville, Md.) were cultured in the absence (control) or presence of TP508 [50 μg/ml](TP pret) for 24 hours. Endothelial cell invasion assays were performed using BD BioCoat™ Angiogenesis System (BD Bioscience, Bedford, Mass.) which utilizes FluoroBlok inserts coated with BD Matrigel Matrix (BD Bioscience, Bedford, Mass.). Control or TP508 pretreated cells were added into the top of the inserts. Medium containing human VEGF [10 ng/ml human recombinant VEGF-A 165aa, R&D System, Minneapolis, Minn.] (V) or medium alone (C) was added to the lower chamber of the insert plate as a chemoattractant to determine angiogenic response to human VEGF. Endothelial cell invasion was performed in normoxic or hypoxic (1% O₂) conditions. After 22 hours of incubation, cells were labeled post-invasion with Calcein AM and measured by detecting the fluorescence of the cells that migrated to the underside of insert membrane.

FIG. 3B shows the effect of TP508 treatment on invasion of endothelial cells toward human VEGF. The results show that control endothelial cells assayed in normoxic conditions or under hypoxic conditions are not stimulated by human VEGF to degrade Matrigel and migrate through the membrane toward human VEGF. In contrast, endothelial cells that were pre-incubated with TP508 show increased invasive properties over control cells that were not pretreated with TP508. In addition, these cells now respond to human VEGF (˜50% more invasion than observed in TP508 pretreated cells without human VEGF and nearly twice as much invasion as control cells toward VEGF). These results demonstrate the ability of TP508 treatment to increase the ability of endothelial cells to respond angiogenically to human VEGF under conditions where non-TP508 treated control cells do not respond at all to human VEGF treatment.

### Example 4

### Effects of TP508 Treatment on Endothelial Cell Invasion and Migration in Response to bFGF

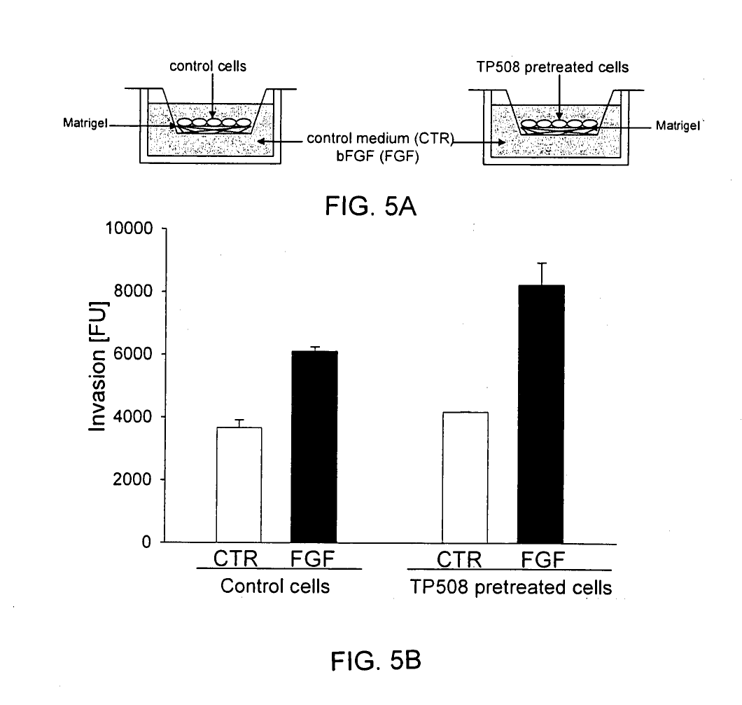

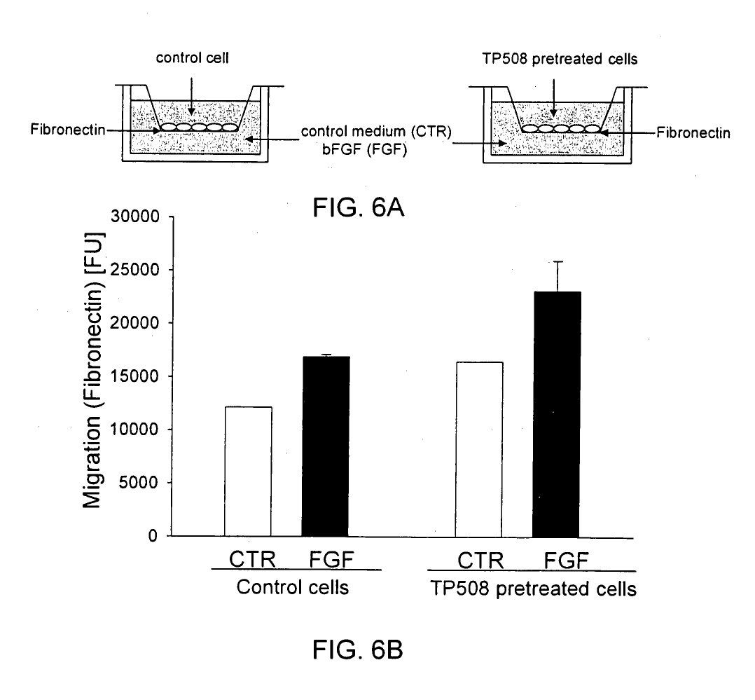

The design of experiments to measure invasion and migration of endothelial cells toward the angiogenic factor bFGF (basic fibroblast growth factor) is shown in FIGS. 5A and 6A, respectively. The standard assay used 5×10⁴ cells added to the top of the insert in 250 μl of medium. The lower portion of the apparatus contained 750 μl of medium, plus or minus bFGF.

Human coronary artery endothelial (HCAE) cells (Lonza Walkersville, Inc., Walkersville, Md.) were cultured in the absence (control cells) or presence of TP508 [50 μg/ml] (TP508 pretreated cells) for 24 hours. Transmembrane cell invasion and migration assays were performed using BD FluoroBlok (BD Bioscience, Bedford, Mass.) inserts coated with BD Matrigel Matrix (a biologically active basement membrane preparation) or with fibronectin, respectively. Control or TP508 pretreated cells were added into the top of the inserts. bFGF [10 ng/ml] (R&D System, Minneapolis, Minn.) (FGF) or medium alone (CTR) were added to the lower chamber of the insert plate as a chemoattractant. The cells were allowed to invade or migrate for 22 hours. Cells were labeled post invasion or post migration with Calcein AM (4 μg/ml) and the fluorescence of the cells that invaded through the BD Matrigel Matrix or migrated to the underside of the insert membrane was measured using a plate reader at 485 nm (excitation) and 530 nm (emission). The results (FIG. 5B) showed that bFGF-induced endothelial cell invasion was ˜170% of the cell invasion observed with control medium (CTR). TP508 pretreated cells showed an increase of bFGF-induced invasion of ˜100% compared to TP508 pretreated cells exposed to control medium (CTR) without bFGF and by ˜125% compared to untreated control cells. Thus, TP508 pretreatment enhanced endothelial invasion toward bFGF relative to untreated control cells.

The results (FIG. 6B) showed that bFGF increased endothelial cell migration through the fibronectin-coated insert by ˜40% compared to control medium not containing bFGF (CTR). TP508 pretreatment increased the basal level of migration towards bFGF by ˜40% compared to the basal level of migration of untreated control cells towards medium without bFGF. Cells pretreated with TP508 showed increased endothelial cell migration toward bFGF by ˜50% compared to control untreated cells. Thus, TP508 enhanced both the basal and bFGF-induced migration in these cells.

### Example 5

### Effects of TP508 Treatment on Endothelial Cell Invasion and Migration in Response to PDGF

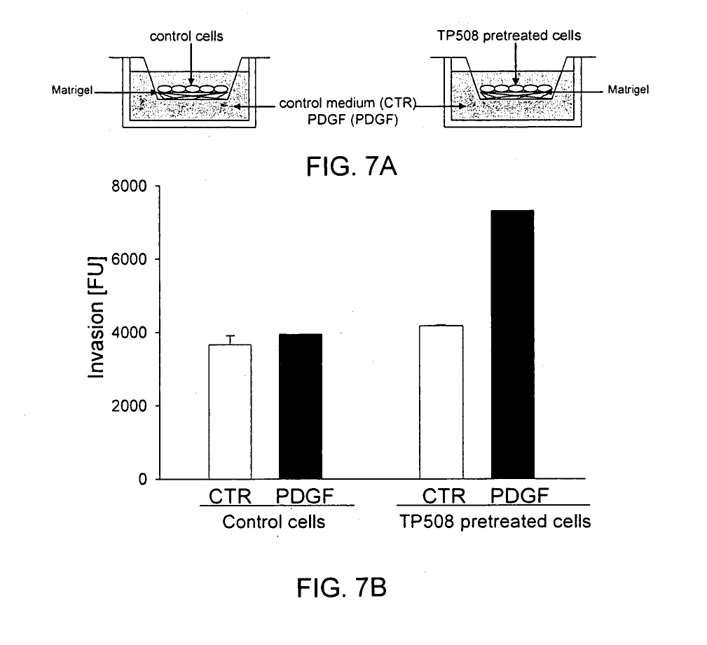

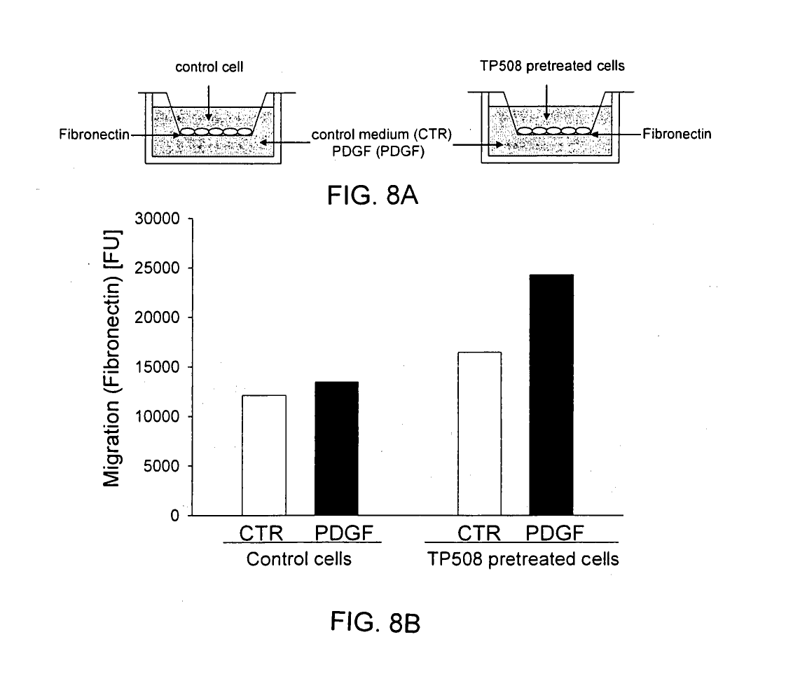

The design of experiments to measure invasion and migration of endothelial cells toward the angiogenic factor PDGF (platelet-derived growth factor-BB) is shown in FIGS. 7A and 8A respectively. The standard assay used 5×10⁴ cells added to the top of the insert in 250 μA of medium. The lower portion of the apparatus contained 750 μl of medium, plus or minus PDGF.

Human coronary artery endothelial (HCAE) cells (Lonza Walkersville, Inc., Walkersville, Md.) were cultured in the absence (control cells) or presence of TP508 [50 μg/ml] (TP508 pretreated cells) for 24 hours. Transmembrane cell invasion and migration assays were performed using BD FluoroBlok inserts coated with BD Matrigel Matrix (a biologically active basement membrane preparation) or with fibronectin (BD Bioscience, Bedford, Mass.), respectively. Control or TP508 pretreated cells were added to the inserts. PDGF [10 ng/ml] (R&D System, Minneapolis, Minn. (PDGF) or medium alone (CTR) were added to the lower chamber of the insert plate as a chemoattractant. The cells were allowed to invade or migrate for 22 hours. Cells were labeled post invasion or post migration with Calcein AM (4 μg/ml) and the fluorescence of the cells that invaded through the BD Matrigel Matrix or migrated to the underside of the insert membrane was measured using a plate reader at 485 nm (excitation) and 530 nm (emission).

The results (FIG. 7B) showed that PDGF had no effect on endothelial cell invasion compared to control medium without added PDGF (CTR). However, TP508 pretreated cells showed increased invasion by ˜75% compared to control (CTR). Thus, TP508 pretreatment enhanced endothelial invasion toward PDGF relative to untreated control cells.

The results (FIG. 8B) showed that PDGF had no effect on endothelial cell migration through the fibronectin-coated insert compared to control (CTR). TP508 pretreatment caused increased endothelial cell migration to PDGF by ˜50% compared to the basal level of migration to PDGF of TP508 pretreated cells. Cells pretreated with TP508 showed 2-fold migration toward PDGF compared to control untreated cells. Thus, TP508 enhanced the basal and PDGF-induced migration in these cells.

1. A method of stimulating cardiac revascularization, vascular endothelial proliferation, or vascular endothelial cell migration in a subject in need thereof, the method comprising administering to the subject a combination in a therapeutically effective amount, the combination comprising one or more angiogenic growth factors, and one or more agonists of the non-proteolytically activated thrombin receptor, wherein the angiogenic growth factor is selected from the group consisting of: human VEGF-A, human VEGF-B, human VEGF-C, human VEGF-D, VEGF-E [Orf virus (D1701)], VEGF-E [Orf virus (NZ2)], VEGF-E_{N27}PlGF, VEGF-E/PlGF, human placental growth factor (PlGF), human platelet derived growth factor D (PDGFD), human platelet derived growth factor alpha (PDGF-α), human platelet derived growth factor 2 (PDGF2), human platelet derived growth factor C (PDGFC), angiogenin, angiopoietin-1, Del-1, acidic fibroblast growth factor (aFGF), basic fibroblast growth factor (bFGF), fibroblast growth factor 4 (FGF 4), follistatin, granulocyte colony-stimulating factor (G-CSF), hepatocyte growth factor (HGF), scatter factor (SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), thymosin beta 4 (T134), connective tissue growth factor, osteopontin, and insulin growth factor (IGF-1).

2. (canceled)

3. The method of claim 1, wherein the combination consists of an angiogenic growth factor and an agonist of the non-proteolytically activated thrombin receptor, wherein the angiogenic growth factor is selected from the group consisting of human VEGF-A, human VEGF-B, human VEGF-C, human VEGF-D, VEGF-E [Orf virus (D1701)], VEGF-E [Orf virus (NZ2)], VEGF-E_{N27}PlGF, VEGF-E/PlGF, human placental growth factor (PlGF), human platelet derived growth factor D (PDGFD), human platelet derived growth factor alpha (PDGF-α), human platelet derived growth factor 2 (PDGF2), human platelet derived growth factor C (PDGFC), angiogenin, angiopoietin-1, Del-1, acidic fibroblast growth factor (aFGF), basic fibroblast growth factor (bFGF), fibroblast growth factor 4 (FGF 4), follistatin, granulocyte colony-stimulating factor (G-CSF), hepatocyte growth factor (HGF), scatter factor (SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), thymosin beta 4 (Tβ4), connective tissue growth factor, osteopontin, and insulin growth factor (IGF-1).

4. The method of claim 1, wherein the combination comprises the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3) and an angiogenic growth factor, wherein the angiogenic growth factor is selected from the group consisting of human VEGF-B, human VEGF-C, human VEGF-D, VEGF-E [Orf virus (D1701)], VEGF-E [Orf virus (NZ2)], VEGF-E_{N27}PlGF, VEGF-E/PlGF, human placental growth factor (PlGF), human platelet derived growth factor D (PDGFD), human platelet derived growth factor alpha (PDGF-α), human platelet derived growth factor 2 (PDGF2), human platelet derived growth factor C(PDGFC), angiogenin, angiopoietin-1, Del-1, acidic fibroblast growth factor (aFGF), basic fibroblast growth factor (bFGF), fibroblast growth factor 4 (FGF 4), follistatin, granulocyte colony-stimulating factor (G-CSF), hepatocyte growth factor (HGF), scatter factor (SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), thymosin beta 4 (Tβ4), connective tissue growth factor, osteopontin, and insulin growth factor (IGF-1).

5-16. (canceled)

17. The method of claim 1, wherein the agonist is a thrombin peptide derivative comprising the amino acid sequence Asp-Ala-R, wherein R is a serine esterase conserved sequence and wherein the thrombin peptide derivative has from about 12 to about 23 amino acids.

18. The method of claim 17, wherein the thrombin peptide derivative comprises an N-terminus which is unsubstituted and a C-terminus which is unsubstituted or a C-terminal amide represented by —C(O)NH₂.

19-20. (canceled)

21. The method of claim 18, wherein the serine esterase conserved sequence comprises the amino acid sequence of Cys-X₁-Gly-Asp-Ser-Gly-Gly-Pro-X₂-Val (SEQ ID NO:15), or a C-terminus truncated fragment of SEQ ID NO:15 having at least six amino acids, wherein X₁ is Glu or Gln and X₂ is Phe, Met, Leu, His or Val and the thrombin peptide derivative comprises the amino acid sequence Arg-Gly-Asp-Ala (SEQ ID NO:16).

22-26. (canceled)

27. The method of claim 21, wherein the thrombin peptide derivative comprises:
i) a polypeptide having the amino sequence of Arg-Gly-Asp-Ala-Cys-X₁-Gly-Asp-Ser-Gly-Gly-Pro-X₂-Val (SEQ ID NO:1), or
ii) the amino acid sequence of Arg-Gly-Asp-Ala-Xaa-X₁-Gly-Asp-Ser-Gly-Gly-Pro-X₇-Val (SEQ ID NO:4),
wherein Xaa is alanine, glycine, serine, or an S-protected cysteine; X₁ is Glu or Gln and X₂ is Phe, Met, Leu, His or Val.

28. The method of claim 27, wherein X₁ is Glu and X₂ is Phe.

29-30. (canceled)

31. The method of claim 21, wherein the amino acid sequence of the thrombin peptide derivative is:
i) Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), an N-terminal truncated fragment of the thrombin peptide derivative having at least fourteen amino acids, or a C-tei ininal truncated fragment of the thrombin peptide derivative having at least eighteen amino acids, or
ii) Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val (SEQ ID NO:5) or a fragment thereof comprising amino acids 10-18 of SEQ ID NO:5.
wherein Xaa is alanine, glycine, serine, or an S-protected cysteine; X₁ is Glu or Gln and X₂ is Phe, Met, Leu, His or Val.

32. The method of claim 1, wherein the agonist is the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3).

33-46. (canceled)

47. The method of claim 1, wherein the agonist is a peptide dimer comprising:
i) two thrombin peptide derivatives which, independently, comprise the amino acid sequence of Arg-Gly-Asp-Ala-Cys-X₁-Gly-Asp-Ser-Gly-Gly-Pro-X₂-Val (SEQ ID NO:1), or
ii) two thrombin peptide derivatives which, independently, are the amino acid sequence Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2) or a fragment thereof comprising amino acids 10-18 of SEQ ID NO:2, wherein X₁ is Glu or Gln and X₂ is Phe, Met, Leu, His or Val.

48. The method of claim 47, wherein the dimer is essentially free of monomer; and the thrombin peptide derivatives are the same and are covalently linked through a disulfide bond.

49-52. (canceled)

53. The method of claim 18, wherein the thrombin peptide derivatives each comprise an N-terminus which is unsubstituted; and a C-terminus which is unsubstituted or a C-terminal amide represented by —C(O)NH₂.

54-59. (canceled)

60. The method of claim 53, wherein X₁ is Glu and X₂ is Phe.

61-63. (canceled)

64. The method of claim 1, wherein the agonist is a peptide dimer represented by the following structural formula:

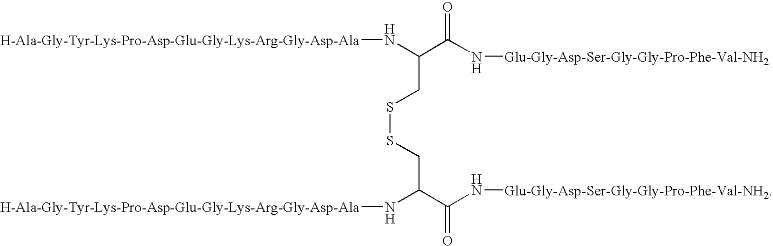

65-83. (canceled)
